(19) United States
(12) Patent Application Publication
(10) Pub. No.: US 2007/0032439 A1
(43) Pub. Date: Feb. 8, 2007

(54) Tarp promoter and uses thereof

(76) Inventor: Magnus Essand, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/542,195

(22) PCT Filed: Jan. 14, 2004
(86) PCT No.: PCT/SE04/00023

§ 371 Date: Apr. 10, 2006

Related U.S. Application Data
(60) Provisional application No. 60/441,688, filed on Jan. 22, 2003.

(51) Int. Cl. A61K 48/00; C12N 15/09

(52) U.S. Cl. 514/44; 435/455

(57) ABSTRACT

A method of providing expression of a molecule in a prostate cell, prostate cancer cell or breast cancer cell comprises introducing into the cell a TARP promoter or a transcriptionally active fragment thereof, operatively linked to a heterologous polynucleotide that codes for the molecule, under conditions effective to direct expression of the molecule in the cell. A method of treating or preventing a prostate-related or breast-related disorder comprises introducing into a cell of a subject in need thereof a TARP promoter, or a transcriptionally active fragment thereof, operatively linked to a heterologous polynucleotide that codes for a molecule capable of killing the cell or capable of preventing the disorder. An isolated polynucleotide sequence comprises a TARP promoter, or a transcriptionally active fragment thereof, operatively linked to a heterologous expressible nucleotide sequence, or operatively linked to other expression control sequences.

### TECHNICAL FIELD

The present invention generally refers to expression of coding sequences and in particular to a tissue-specific promoter and uses thereof.

### BACKGROUND

Prostate cancer remains the most common solid tumor and the second leading cause of cancer-related deaths among men in the USA. An estimated 180,400 new cases and 31,900 deaths related to prostate cancer were expected for 2000 (Cookson, Cancer Control, 8: 133-140, 2001). The current standard therapies employed for organ-confined prostate cancer include external beam irradiation or surgery, in some circumstances incorporating neoadjuvant or adjuvant hormonal therapy. While these therapies are relatively effective in the short term, a significant proportion of patients that initially present localized disease ultimately relapse. For metastatic prostate cancer the main therapy is androgen ablation. While this provides cytoreduction and palliation, progression to hormone-refractory disease typically occurs within the order of 14-20 months. A great number of clinical research studies have been reported in the field of chemotherapy for advanced androgen-independent prostate cancer. So far, no combination of chemotherapy reported has improved the overall survival of patients. Thus, there is an urgent need for finding effective methods to control localized prostate cancer and treat advanced prostate cancer, which can replace or complement the prior art techniques.

Cancer gene therapy strategies that attempt to exploit the biological uniqueness of each particular tumor represent a promising novel form of therapy. Such strategies may be based on promoter-controlled therapeutic gene expression. A vector, e.g. a recombinant virus, with an expression cassette where a tissue-specific promoter regulates expression of a therapeutic gene can be used to kill cancer cells derived from the very same tissue. Prostate cancer is a particularly appropriate target for such approach since the human prostate is an accessory organ that is non-essential for life and is neither required for potency or urinary continence.

A number of therapeutic approaches relying upon prostate-specific transcriptional elements have been envisioned, including therapeutic genes expressed under the control of prostate-specific regulatory sequences and therapeutic viruses whose replication is limited to prostate cells. For example, usage of the promoter and enhancer elements for prostate specific antigen (PSA), kallikrein 2 (hK2), prostate-specific membrane antigen (PSMA), osteocalcin and rat probasin have been described in WO 95/19434, WO 96/14875, WO 00/12763, WO 00/14234, WO 00/52156, WO 01/27256, WO 01/32685, WO 01/70175 and U.S. Pat. No. 5,952,488.

### SUMMARY

It is a general object of the present invention to provide a tissue-specific promoter.

It is another object of the invention to provide a regulatory sequence enabling expression of molecules, including therapeutic molecules, in a tissue-specific manner.

Yet another object of the invention is to provide a vector with a regulatory sequence for use in gene therapy against prostate-related disorders, including prostate cancer, in male patients and/or breast-related disorders, including breast cancer, in female patients.

These and other objects are met by the invention as defined by the accompanying patent claims.

Briefly, the present invention involves the TARP promoter and uses thereof, e.g. for obtaining tissue-specific expression of molecules, including therapeutic molecules. The TARP promoter is the expression control sequence of two mRNA transcripts from the non-rearranged T cell receptor (TCR) γ chain locus. The transcripts are uniquely expressed in normal luminal epithelial cells of the prostate and prostate cancer cells in males and in breast cancer cells in females. In γδ T cells, the TARP promoter sequence is absent due to rearrangements of TCR γ chain gene segments, whereas it is inactive in cells other than the above-identified prostate and cancer cells. The TARP promoter sequence comprises an androgen response element (ARE) enabling binding of activated androgen receptor (AR) thereto. Upon AR binding to the ARE in the promoter sequence, TARP mRNA transcription is induced. The about 200 nucleotides directly upstream of the TARP mRNA transcription initiation site constitutes the proximal TARP promoter, whereas the large TARP promoter includes the about 2640 nucleotides upstream of the TARP mRNA transcription initiation site. Both the proximal and large TARP promoters are specifically active in normal prostate epithelial cells, prostate cancer cells and prostate cancer cell lines and to a lesser degree in breast cancer cell lines. In prostate cancer cell lines the TARP promoter is inducible by testosterone.

The invention also involves recombinant regulatory sequences comprising the TARP promoter (TARPp), or transcriptionally active fragments thereof, operatively linked to other expression control sequences, such as the enhancer sequence for the gene encoding prostate specific antigen (PSA) and/or the enhancer sequence for the gene encoding prostate specific membrane antigen (PSMA). A recombinant regulatory sequence comprising the TARP promoter and the PSA enhancer (PSAe/TARPp) is highly prostate-specific and is strictly controlled by testosterone. Luciferase reporter gene assays, using the prostate adenocarcinoma cell line, LNCaP, demonstrates that the PSAe/TARPp regulatory sequence has approximately 20 times higher transcriptional activity than a regulatory sequence comprising the PSA promoter and the PSA enhancer (PSAe/PSAp), which is one of the most widely employed regulatory sequence in prostate cancer gene therapy. Another highly prostate-specific recombinant regulatory sequence comprises the TARP promoter and the PSMA enhancer is (PSMAe/TARPp). The PSMA enhancer is not dependent on testosterone for activity. The transcriptional activity of PSMAe/TARPp is more than 600 times higher than the prior art PSAe/PSAp regulatory sequence in LNCaP cells cultured in the absence of testosterone. A recombinant regulatory sequence comprising the TARP promoter, the PSMA enhancer and the PSA enhancer (PSAe/PSMAe/TARPp) has high transcriptional activity in prostate cancer cell lines and does not exert transcriptional activity in cell lines of non-prostate origin. The PSAe/PSMAe/TARPp sequence is not dependent on testosterone for transcriptional activity although testosterone increases its activity somewhat. Since prostate cancer patients are often treated by androgen withdrawal it may be beneficial to the patient to use a regulatory sequence in gene therapy with high prostate-specific gene expression also in the absence of androgens, such as the PSAe/PSMAe/TARPp sequence.

For high and specific therapeutic gene expression in breast cancer cells the TARP promoter is preferably combined with other expression control sequences, such as an enhancer for a gene encoding a breast-specific protein, which preferably is upregulated in response to estrogens.

A regulatory sequence comprising the TARP promoter, or a transcriptionally active fragment thereof, either alone or operatively linked to other expression control sequences, such as the PSA enhancer and the PSMA enhancer, may be operatively linked to a heterologous nucleotide sequence for expression thereof in only prostate cells, prostate cancer cells and breast cancer cells. The heterologous nucleotide sequence preferably codes for one or several therapeutic molecules effective in treating or delaying prostate cancer in male patients or breast cancer in female patients.

The invention also involves a vector, e.g. a recombinant adenovirus vector, with a regulatory sequence comprising the TARP promoter of the invention or a transcriptionally active fragment thereof, either alone or operatively linked to other expression control sequences, where the regulatory sequence is operatively linked to an expressible nucleotide sequence for use in treatment or prevention of a prostate-related disorder in males, including prostate cancer, and/or a breast-related disorder in females, including breast cancer. The said vector may be used for, but are not limited to: suicide gene therapy (expression of prodrug), induction of apoptosis (expression of proapoptotic molecule), toxin expression, radioisotopic therapy (expression of iodide symporter), immunotherapy (expression of immunomodulatory molecule), expression interference (expression of a molecule that interfere with the expression machinery of a molecule essential for cell survival), and/or expression of a viral gene essential for viral replication, due to the tissue-specific activity of the TARP promoter.

Furthermore, the invention involves methods for delivering a molecule and providing expression of said molecule, e.g. a therapeutic molecule, in a prostate cell, prostate cancer cell or breast cancer cell in vitro, or in vivo in a subject, preferably a human patient. The methods comprise introducing into a cell the TARP promoter of the invention or a transcriptionally active fragment thereof, either alone or together with other expression control sequences, operatively linked to a heterologous polynucleotide sequence that codes for the molecule, under conditions effective in providing expression of the molecule. The TARP promoter of the invention is preferably included in a vector for facilitating introduction thereof in the cell.

The invention also involves methods for treating, involving inhibiting or delaying, or preventing a prostate-related disorder, including prostate cancer, in male patients and breast-related disorder, including breast cancer, in female patients. The methods involve introducing into a cell of the patient a vector comprising the TARP promoter of the invention or a recombinant regulatory sequence comprising the TARP promoter, operatively linked to a heterologous polynucleotide that codes for a molecule capable of killing the cell or preventing the disorder.

In addition, the invention involves use of the TARP promoter for the manufacturing of a medicament for the treatment of a prostate-related disorder, including prostate cancer, in males, or a breast-related disorder, including breast cancer, in females. The treatment includes the therapeutic and/or prophylactic treatment of the disorder. The medicament then comprises a vector including the TARP promoter preferably operatively linked to a heterologous polynucleotide that codes for a molecule capable of preventing the disorder, e.g. by killing the cancer cells.

The invention offers the following advantages:
Provides highly tissue-specific transcriptional activity that enables expression of heterologous nucleotide sequences, including sequences coding for therapeutic molecules, exclusively in prostate cells and prostate cancer cells in males and breast cancer cells in females;
May be combined with several other expression control sequences without loss of tissue specificity;
May be combined with enhancer sequences to enable high activity also under androgen-deprived conditions, i.e. in the case of hormone refractory prostate cancer; and
Have much higher activity than most prior art employed prostate-specific promoter sequences.

Other advantages offered by the present invention will be appreciated upon reading of the below description of the embodiments of the invention.

### SHORT DESCRIPTION OF THE DRAWINGS

The invention together with further objects and advantages thereof, may best be understood by making reference to the following description taken together with the accompanying drawings, in which:

FIG. 1 is a schematic illustration of the human T cell receptor γ chain locus with the position and orientation of the TARP promoter indicated by a triangle;

FIG. 2 is a schematic illustration of the TARP mRNA transcripts specifically expressed by normal luminal epithelial cells of the prostate, prostate cancer cells and breast cancer cells;

FIG. 3 is an illustration of the human proximal TARP promoter sequence;

FIG. 4 is an illustration of the human large TARP promoter sequence;

FIG. 5 is an illustration of the human TARP enhancer sequence;

FIG. 6 is an illustration of the human PSA enhancer sequence;

FIG. 7 is an illustration of the human PSMA enhancer sequence;

FIG. 8 is an illustration of the sequence of the mouse H19 DNA insulator;

FIG. 9 is an illustration of a Northern blot analysis of TARP mRNA expression in LNCaP cells cultured in steroid-depleted medium with or without addition of synthetic testosterone (R1881);

FIG. 10 is an illustration of a RT-PCR analysis of TARP mRNA expression in LNCaP cells cultured in steroid-depleted medium with or without addition of synthetic testosterone (R1881), with or without actinomycin-D (Act D), and with or without cyclohexamide (CHX);

FIG. 11 is an illustration of homologies between different androgen response element (ARE) sequences;

FIG. 12 is an illustration of the result from a luciferase reporter gene assay showing relative transcriptional activity for different portions of the 5′ flanking sequence of the TARP (T) gene in LNCaP cells;

FIG. 13 is an illustration of the result from a luciferase reporter gene assay showing testosterone inducibility of transcription for different portions of the 5′ flanking sequence of the TARP (T) gene in LNCaP cells;

FIG. 14 is an illustration of an electrophoretic mobility shift assay (EMSA) showing specific binding of the androgen receptor (AR) to the wild-type androgen response element (ARE) probe sequence at position −186 in the TARP promoter. The AR binding is abolished by a 100-fold excess of non-labeled wild-type ARE competitor. AR binding does not occur to a mutated ARE sequence;

FIG. 15 is an illustration of a DNA precipitation/Western blot analysis showing specific binding of the androgen receptor (AR) to the wild-type androgen response element (ARE) sequence at position −186 in the TARP promoter while binding does not occur to a mutated ARE sequence;

FIG. 16 is an illustration of the result from a luciferase reporter gene assay comparing the relative transcriptional activity of the PSA promoter (PSAp) with the TARP promoter (TARPp) in LNCaP and PC346-C cells cultured in steroid-depleted medium with (10 nM R1881) or without (no stimuli) addition of testosterone;

FIG. 17 is an illustration of the result from a luciferase reporter gene assay comparing the relative transcriptional activity of a recombinant regulatory sequence composed of the PSA enhancer and the PSA promoter (PSAe/PSAp) with a recombinant regulatory sequence composed of the PSA enhancer and the TARP promoter (PSAe/TARPp), in LNCaP and PC346-C cells cultured in steroid-depleted medium with (10 nM R1881) or without (no stimuli) addition of testosterone;

FIG. 18 is an illustration of the result from a luciferase reporter gene assay comparing the relative transcriptional activity of the recombinant regulatory sequences comprising either PSAe/TARPp, the PSMA enhancer and TARP promoter (PSMAe/TARPp), the PSMA enhancer, PSA enhancer and TARP promoter (PSMAe/PSAe/TARPp) and the PSA enhancer, PSMA enhancer and TARP promoter (PSAe/PSMAe/TARPp) in LNCaP and PC346-C cells cultured in steroid-depleted medium with (10 nM R1881) or without (no stimuli) addition of testosterone;

FIG. 19 is an illustration of the result from a luciferase reporter gene assay with the relative transcriptional activity of the recombinant PSAe/PSMAe/TARPp regulatory sequence in cell lines of different origins, cultured in steroid-depleted medium;

FIG. 20 is an illustration of the result from a luciferase reporter gene assay comparing the relative transcriptional activity of a recombinant adenovirus (Ad) with an expression cassette containing the PSAe/PSMAe/TARPp (PPT) regulatory sequence and the luciferase (Luc) reporter gene, Ad[PPT-Luc], with a recombinant adenovirus with an H19 insulator (1) to shield the expression cassette from upstream adenoviral sequences Ad[I/PPT-Luc];

FIG. 21 is an illustration of the result from a luciferase reporter gene assay comparing the relative transcriptional activity of Ad[PPT-Luc] with Ad[I/PPT-Luc] in cell lines of different origins, cultured in steroid-depleted medium; and

FIG. 22 is a schematic illustration of a recombinant adenovirus vector with an H19 insulator-shielded expression cassette where the recombinant PSA enhancer/PSMA enhancer/TARP promoter regulatory sequence controls expression of a reporter gene or therapeutic gene.

### DETAILED DESCRIPTION

The invention generally refers to the promoter sequence of TARP and uses thereof.

Unless defined otherwise, all technical and scientific terms used herein have the meaning commonly understood by a person skilled in the art to which the present invention belongs.

The following references provide a general definition of many of the terms used in this invention: Singleton et al., Dictionary of microbiology and molecular biology (3^{rd} ed., 2002); The Cambridge dictionary of science and technology, (Walker ed., 1988); The glossary of genetics, (Rieger et al., eds., 5th ed., 1991); Hale and Marham, Harper Collins dictionary of biology, 1991; and Lewin, Genes VII (7th ed., 1999). For clarity of the invention, the following definitions are used herein.

“Polynucleotide” or “nucleic acid” refers to a polymer composed of nucleotide units (ribonucleotides, deoxyribonucleotides, related naturally occurring structural variants, and/or synthetic non-naturally occurring analogs thereof) linked via phosphodiester bonds, related naturally occurring structural variants, and/or synthetic non-naturally occurring analogs thereof. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C), in which “U” replaces “T”. The term polynucleotide includes, unless otherwise specified, double stranded and single stranded DNA, cDNA and RNA. Also hybrids such as DNA-RNA hybrids are included in the term. Reference to a polynucleotide or nucleic acid sequence can also include modified bases known to the person skilled in the art.

“cDNA” refers to a DNA that is complementary or identical to an mRNA, in either single stranded or double stranded form.

“Recombinant nucleic acid” refers to a nucleic acid having nucleotide sequences that are not naturally joined together. A recombinant nucleic acid may serve a coding function (fusion of genes/gene fragments) or a non-coding function (e.g. promoter, origin of replication, ribosome-binding sites etc.).

“Polypeptide” refers to a polymer composed of amino acids residues, related naturally occurring structural variants, and/or synthetic non-naturally occurring analogs thereof linked via peptide bonds, related naturally occurring structural variants, and/or synthetic non-naturally occurring analogs thereof. The term “protein” typically refers to large polypeptides. The term “peptide” typically refers to short polypeptides.

The term “encoding” or “coding for” refers to the inherent property of specific sequences of nucleotides in a polynucleotide, such as a gene, a cDNA or an mRNA, to serve as templates for synthesis of other molecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA produced by that gene produces the protein in a cell or other biological (in vivo and in vitro) system. Both the coding strand, the nucleotide sequence of which is identical to the mRNA sequence and is usually provided in sequence listings, and the non-coding strand, used as the template for transcription of a gene or cDNA can be referred to as encoding the protein or other product of that gene or cDNA. Nucleotide sequences that encode proteins and RNA may include introns.

The term “coding sequence” or “coding nucleotide sequence” refers to a polynucleotide with the properties of being able to be transcribed into either a defined sequence of nucleotides (tRNA, rRNA and mRNA) and, in the case of mRNA transcription, being further translated into a polypeptide. The coding sequence may be a gene, a cDNA or a recombinant nucleic acid.

The term “TARP” refers to the T cell receptor (TCR) γ chain alternate reading frame protein that in males is uniquely expressed in epithelial cells within the acinar ducts of the prostate and in prostate cancer cells. TARP expression has also been detected in male prostate cancer cell lines and female breast cancer cell lines.

The term “TARP mRNA transcript(s)” refers to two mRNA transcripts of approximately 1.1 and 2.8 kb in size comprising the major portion of the Jγ1.2 gene segment and the three exons of the Cγ1 gene segment. The two TARP mRNA transcripts originate from the same transcription initiation site. The difference in size is due to that two polyadenylation signals (AATAAA) at different location, downstream of the Cγ1 gene segment, are used for transcriptional termination.

“Promoter” is the minimal nucleotide sequence required to direct transcription. The promoter may include elements that render the promoter-depending gene expression cell-type or tissue specifically controllable or inducible by external signals or agents.

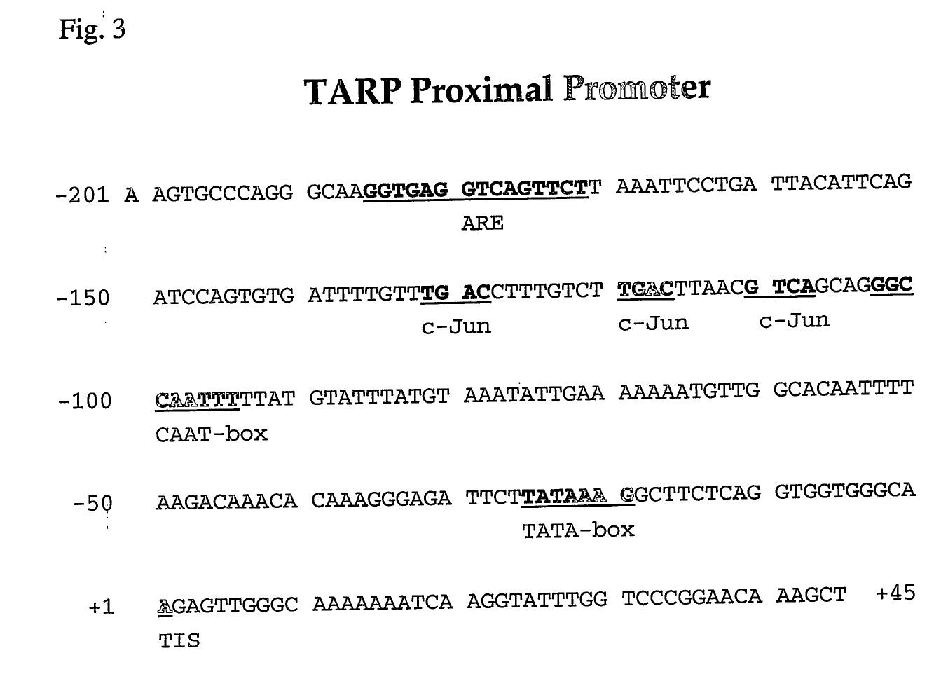

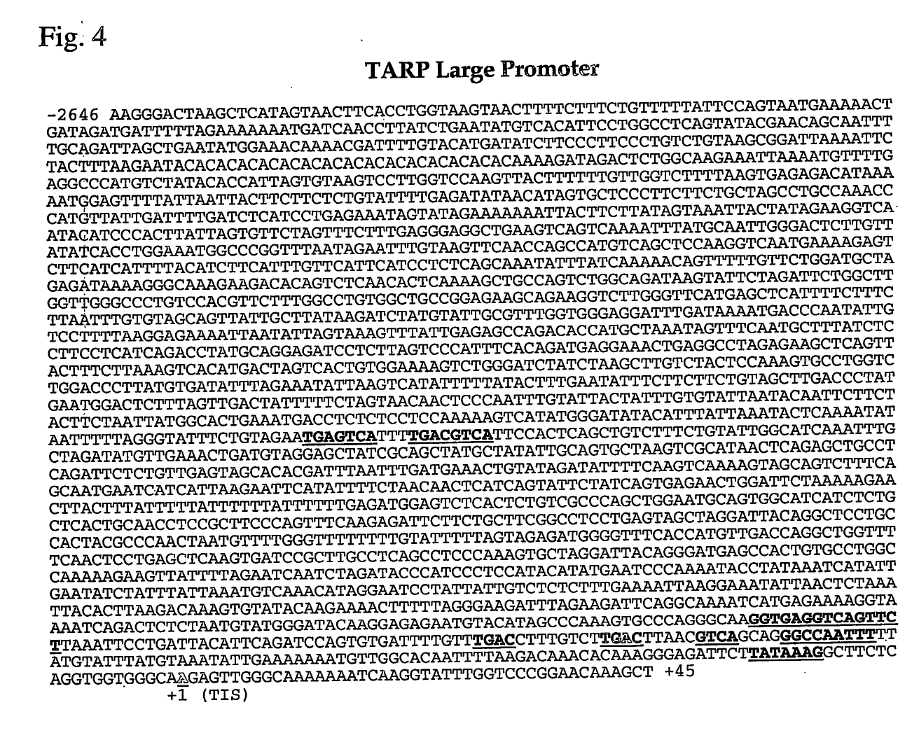

The term “TARP promoter”, as used herein, includes both the proximal TARP promoter and the large TARP promoter. The “proximal TARP promoter” refers to a nucleotide sequence of sequence SEQ ED NO: 1 (FIG. 3), from nucleotide 1 to 201 (−201 to +1, relative the TARP transcription initiation site (TIS) in FIG. 3), as well as complementary sequences and sequences which exhibit at least about 80% sequence identity, e.g. at least 85% sequence identity, preferably at least about 90% sequence identity, more preferably at least 95%, e.g. at least 98% sequence identity with SEQ ID NO: 1 from nucleotide 1 to 201, e.g. the sequence of SEQ ID NO: 1. The sequence identity of two polynucleotides may be determined by several different methods known to the person skilled in the art including, but not limited to, BLAST program of Altschul et al. (J. Mol. Biol., 215: 403-410, 1990). The SEQ ID NO: 1 is a sequence example of the proximal TARP promoter of the invention. The term “large TARP promoter” refers to a nucleotide sequence of sequence SEQ ID NO: 2 (FIG. 4), from nucleotide 1 to 2646 (−2646 to +1, relative to the TARP transcription start point in FIG. 4), as well as complementary sequences and sequences which exhibit at least about 80% sequence identity, e.g. at least 85% sequence identity, preferably at least about 90% sequence identity, more preferably at least about 95%, e.g. at least 98% sequence identity with SEQ ID NO: 2 from nucleotide 1 to 2646, e.g. the sequence of SEQ ID NO: 2.

The term “transcriptionally active fragment” of the TARP promoter refers to a nucleotide sequence of the TARP promoter having sequence elements adapted for directing transcription of an operatively linked polynucleotide sequence. Such a fragment preferably comprises at least one of the sequence elements of the TARP promoter, e.g. the TATA box positioned at nucleotides −26 to −20 relative the TARP transcription initiation site (see FIGS. 3 and 4), the androgen response element (ARE) at position −186 to −172, the CAAT box at position −103 to −95 or one of the c-Jun binding sites at positions −132 to −129, −120 to −117 and −111 to −108, respectively, or complementary sequences and sequences which exhibit at least about 80% sequence identity, e.g. at least 85% sequence identity, preferably at least about 90% sequence identity, more preferably at least about 95%, e.g. at least 98% sequence identity of the above-identified sequence elements. The transcriptionally active fragment preferably includes at least the TATA box and/or ARE, and more preferably also comprises the CAAT box, such as a sequence that comprises the TATA box, ARE, CAAT box and at least one of the c-Jun binding sites identified above. The sequence elements are preferably positioned within the transcriptionally active fragment in the order found in the TARP promoter. In addition, the nucleotide distances between the sequence elements in the transcriptionally active fragment are preferably substantially similar, such as less than ±10 nucleotides, preferably less than ±7 nucleotides, more preferably less than ±5 nucleotides, e.g. less than ±2 nucleotides or less than ±1 nucleotide, to the corresponding nucleotide distances between the sequence elements of the TARP promoter (FIGS. 3 and 4, SEQ ID NO: 1 and 2). The distances are more preferably the same as the corresponding distances in the TARP promoter. An example of a transcriptionally active fragment of the TARP promoter of the invention comprises an ARE at position −186 to −172, and a TATA box at position −26 to −20, preferably also a CAAT box at position −103 to −95, more preferably also three c-Jun binding sites at positions −132 to −129, −120 to −117 and −111 to −108, relative the transcription initiation site. The nucleotides found in other positions of the transcriptionally active fragment than the above described sequence elements may be any of A, T, C or G, preferably the respective nucleotides found in the sequence of FIGS. 3 and 4.

“Androgen response element (ARE)” refers to a 15 nucleotide long imperfect palindromic sequence consisting of two six base pair half-sites that are separated by a three nucleotide long spacer. The ARE sequence is recognized by the androgen receptor (AR) as well as the glucocorticoid receptor and the progesterone receptor. The expressions “TARP promoter ARE” and “ARE[−186]” refer to the ARE sequence of GGTGAGGTCAGTTCT positioned at nucleotides −186 to −172 in the TARP promoter relative to the transcription initiation site.

The “transcription initiation site (TIS)” and “transcription start point” refer to the first nucleotide in an RNA transcript. The “TARP transcription initiation site” refers to the first nucleotide of the TARP mRNA transcripts, which is depicted +1, in FIGS. 3 and 4, +5 nucleotides upstream or downstream of nucleotide +1.

“Enhancer” refers to a regulatory sequence that increases expression of an operatively linked gene or coding sequence but does not have promoter activity. An enhancer can generally be provided upstream, downstream and to the other side of a promoter without significant loss of activity. Furthermore, an enhancer may be positioned within the coding sequence of the gene.

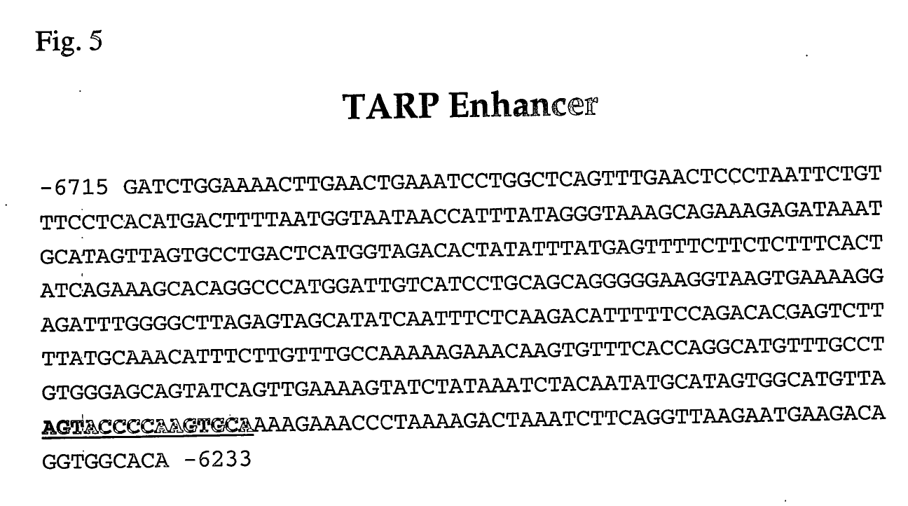

The expression “TARP enhancer” refers to the nucleotide sequence of SEQ ID NO: 3 (FIG. 5), as well as complementary sequences and sequences which exhibit at least about 80% sequence identity, e.g. at least 85% sequence identity, preferably at least about 90% sequence identity, more preferably at least about 95%, e.g. at least 98% sequence identity with SEQ ID NO: 3.

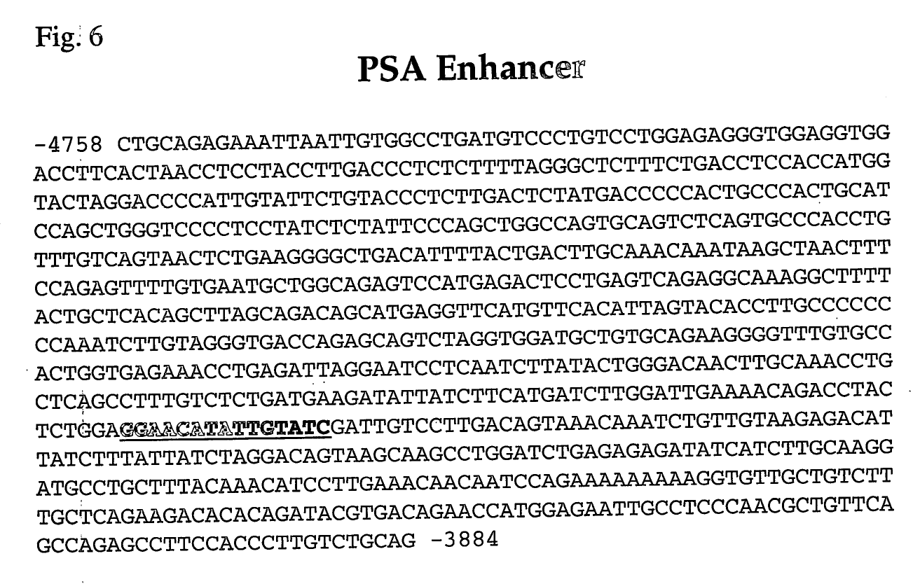

The term “PSA enhancer” refers to the nucleotide sequence of SEQ ID NO: 4 (FIG. 6) as well as complementary sequences and sequences which exhibit at least about 80% sequence identity, e.g. at least 85% sequence identity, preferably at least about 90% sequence identity, more preferably at least about 95%, e.g. at least 98% sequence identity with SEQ ID NO: 4.

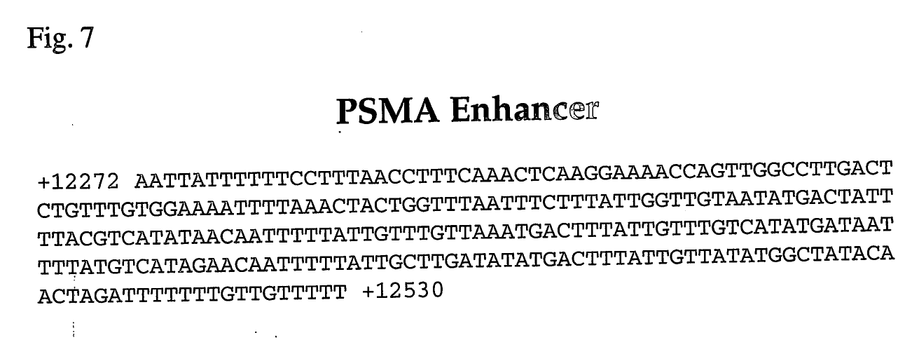

The expression “PSMA enhancer”, refers to the nucleotide sequence of SEQ ID NO: 5 (FIG. 7) as well as complementary sequences and sequences which exhibit at least about 80% sequence identity, e.g. at least 85% sequence identity, preferably at least about 90% sequence identity, more preferably at least about 95%, e.g. at least 98% sequence identity with SEQ ID NO: 5.

The term “DNA insulator” refers to a nucleic acid sequence with the ability to block the action of enhancers, protect against position effects and/or prevent gene activation.

“Expression control sequence”, “expression control element”, “regulatory sequence” or “regulatory element” refers to a nucleotide sequence in a polynucleotide that regulates the expression (transcription and/or translation) of a nucleotide sequence operatively linked thereto. A regulatory sequence may include a promoter, an enhancer, a silencer, a transcription or a translation start site and/or a transcription or translation termination region.

The term “operatively linked” or “operably associated” refers to a functional linkage—between the regulatory sequence and a coding sequence or a functional linkage between two regulatory sequences. The components so described are thus in a relationship permitting them to function in their intended manner. By placing a coding sequence under regulatory control of a promoter or another regulatory sequence means positioning the coding sequence such that the expression of the coding sequence is controlled by the regulatory sequence.

“Expression cassette” refers to a recombinant nucleic acid construct comprising a regulatory sequence operatively linked to an expressible nucleotide sequence.

“Expression vector” refers to a vector comprising an expression cassette. Expression vectors include all those known in the art, such as cosmids, plasmids (e.g. naked or in complex with liposome or polymer) and viruses that incorporate the expression cassette.

The term “transgene” refers to a polynucleotide sequence with the properties of being able to be transcribed into either a defined sequence of nucleic acids (tRNA, rRNA and mRNA) and, in the case of mRNA transcription, being further translated into a polypeptide. The coding sequence may be a gene, a cDNA or a recombinant nucleic acid. It also refers to any nucleic acid sequence that is inserted by artifice into a cell and becomes part of the genome of the cell and preferably of an organism developing from that cell. The sequence may either be stably integrated or provided as a stable extrachromosomal element.

A “therapeutic gene” is a polynucleotide sequence which codes for a “therapeutic molecule” in forms of a defined sequence of nucleic acids (i.e., rRNA, tRNA and mRNA) or a defined sequence of amino acids which, when expressed, can be used to treat the cause, or ameliorate, by lessening the detrimental effect of, the symptoms of a disorder. Thus a therapeutic gene may code for a therapeutic molecule in form of a short interfering RNA, antisense RNA, ribozyme or polypeptide.

The term “reporter gene” refers to a gene or coding sequence encoding an easily measurable phenotype that can be used to measure gene expression. Examples of reporter genes are chloramphenicol acetyl transferase (CAT), chromagenic genes, e.g. the β-galactosidase gene (lacZY), β-galacturoindase gene (gusA) and catechol monooxygease gene (xylE), fluorescence genes, e.g. the green fluorescent protein gene (gfp), yellow fluorescent protein gene (yfp), blue fluorescent protein gene (bfp) and red fluorescent protein gene (rfp) and luminescence genes, e.g. the bacterial luciferase gene (lux), firefly luciferase gene (luc) and renilla luciferase.

Description of the TARP Promoter

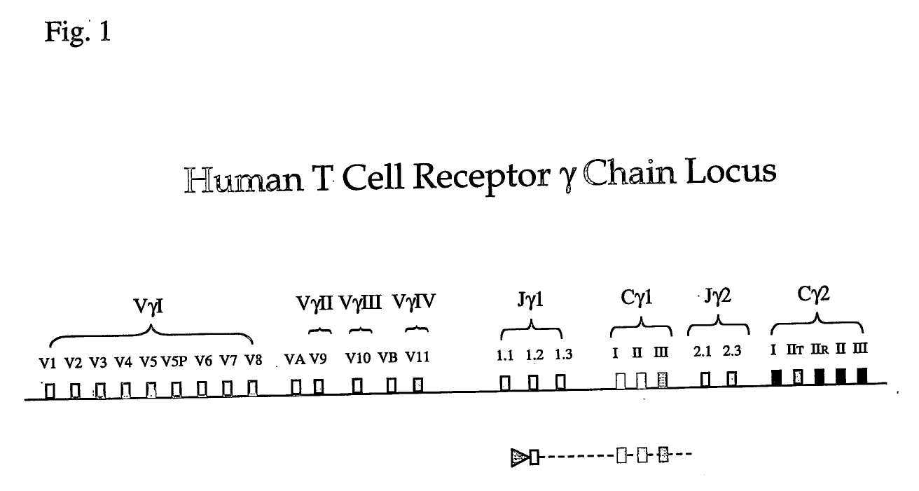

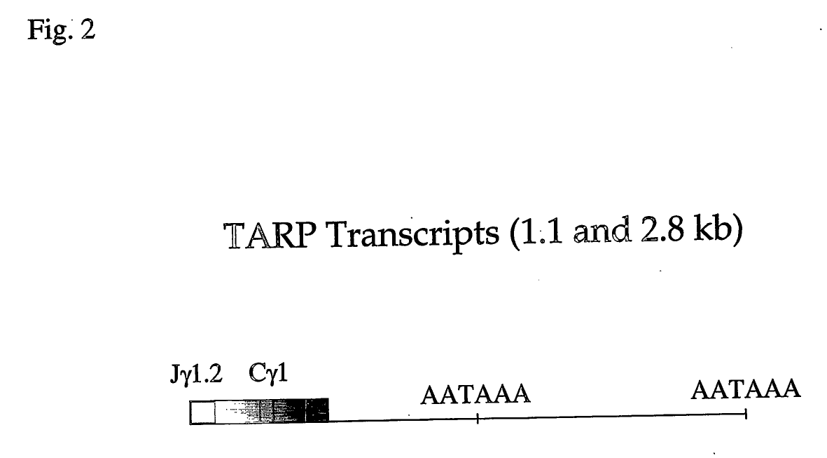

TARP is a recently identified protein that in males is uniquely expressed in luminal epithelial cells within the acinar ducts of the human prostate, in benign prostatic hyperplasia cells and in prostate cancer cells. TARP expression has also been detected in male prostate cancer cell lines and female breast cancer cell lines. The TARP mRNA transcripts originate from the human T cell receptor (TCR) 7 chain locus on chromosome 7, p15-p14, which is schematically illustrated in FIG. 1. This locus comprises several variable (V), joining (J) and constant (C) gene segments that undergo a series of rearrangements to form functionally active genes in mature γδ T-lymphocytes. In humans 14 Vγ gene segments have been identified and divided into four separate subgroups (VγI, VγII, VγIII and VγIV) based on sequence homology. There are five Jγ gene segments organized in two subgroups (Jγ1 and Jγ2) and two different Cγgenes (Cγ1 and Cγ2). The gene rearrangements in γδ T cells link a variable gene segment to one of the joining gene segments prior to the transcription. Thus, due to the gene rearrangement, the TARP promoter sequence is absent in mature γδ T cells. However, in prostate and breast epithelial cells no such gene rearrangement occurs and a functional TARP promoter is present. The TARP mRNA transcripts comprise the Jγ1.2 gene segment, the three exons of the Cγ1 gene segment and various length of untranslated 3′end sequence as illustrated in FIG. 2. For more information of TARP, reference is made to Essand et al., Proc. Natl. Acad. Sci. USA, 96: 9287-9292, 1999; Wolfgang et al., Proc. Natl. Acad. Sci. USA, 97: 9437-9442, 2000; and Wolfgang et al., Cancer Res., 61: 8122-8126, 2001.

The transcription initiation site of the TARP transcripts is located in the TCR Jγ1.2 gene segment and the TARP promoter sequence is located in and mainly directly upstream of the TCR Jγ1.2 gene segment. The TARP promoter sequence is transcriptionally active in prostate cells, prostate cancer cells and breast cancer cell. So far no other human cell type that provides transcriptional activity of the TARP promoter has been identified. Therefore, the TARP promoter is a promising candidate for expression of therapeutic genes in prostate cancer and breast cancer cells.

The inventor has characterized the TARP promoter and showed that it provides the tissue-specific expression of TARP. FIG. 3 is an illustration of the sequence of a proximal TARP promoter including the about 200 nucleotides directly upstream of the transcription initiation site (TIS), depicted +1 in FIGS. 3 and 4. This proximal TARP promoter contains sequence elements or motifs for general transcription factors and inducible transcription factors involved in initiation and regulation of transcription. These motifs include a TATA box located at −26 relative to the TIS of TARP, a CAAT box at −103, three c-Jun binding sites at −111, −120 and −132 and a functional androgen response element (ARE) at position −186, depicted in bold and underlined in FIG. 3.

A similar illustration of the large TARP promoter comprising about 2645 nucleotides upstream of the TIS of TARP is illustrated in FIG. 4. In addition to the DNA motifs for the transcription factors identified for the proximal TARP promoter, the large TARP promoter also comprises a transcription response region identified at −1100, with a cAMP response element (CRE), TGACGTCA, and an activator protein-1 (AP-1) response element, TGAGTCA, depicted in bold and underlined in FIG. 4.

An enhancer sequence is situated upstream of the TARP promoter, at −6715 to −6233 relative to the nS of TARP. This enhancer sequence is illustrated in FIG. 5, with an ARE at position −6301, depicted in bold and underlined.

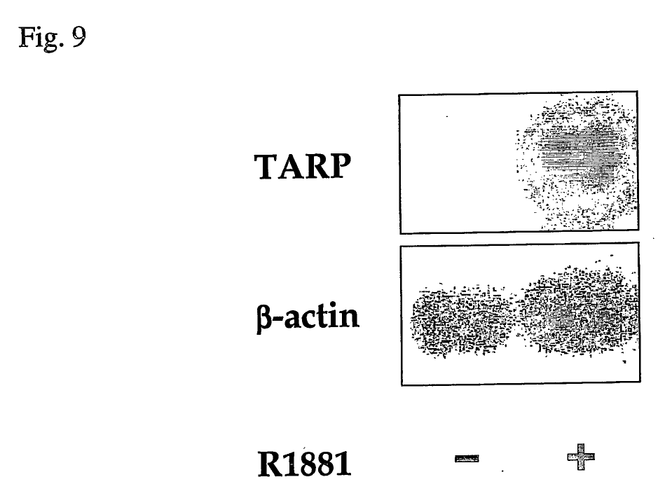

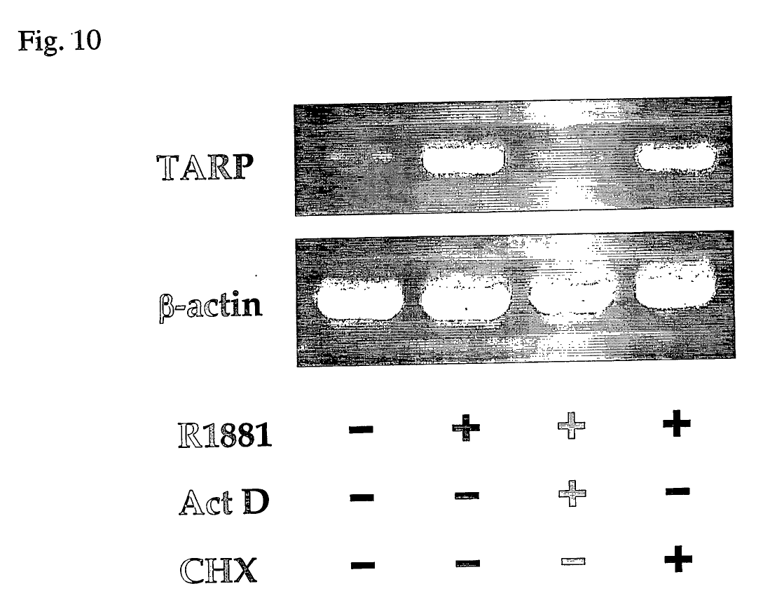

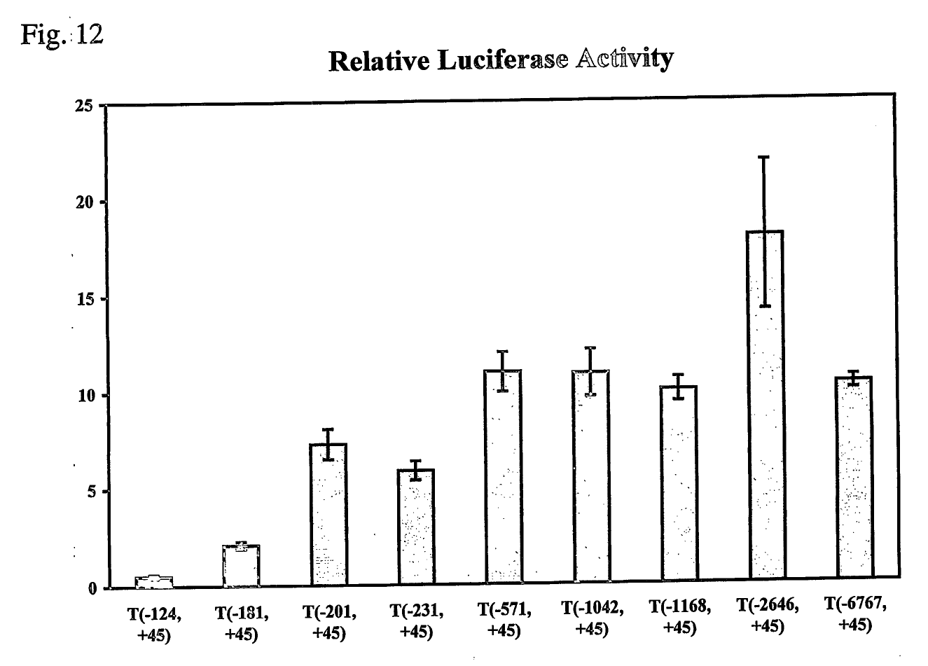

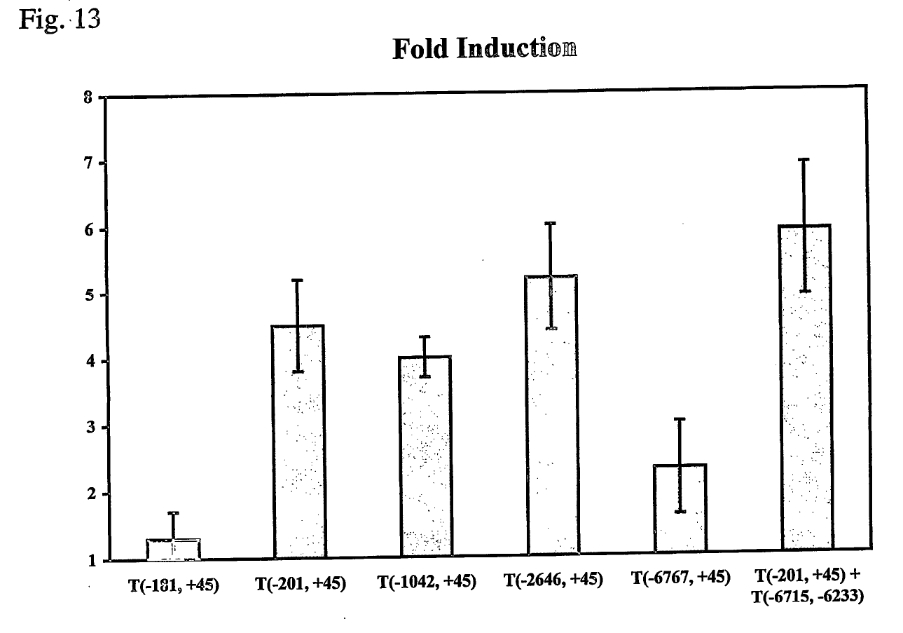

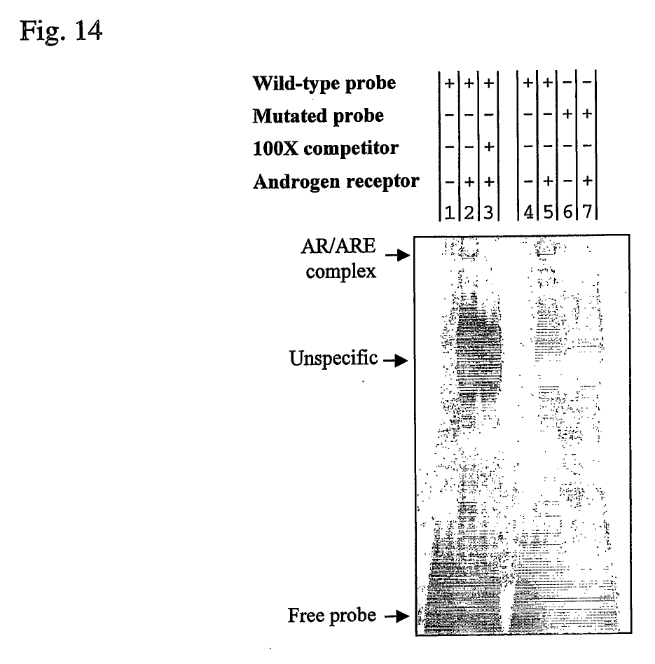

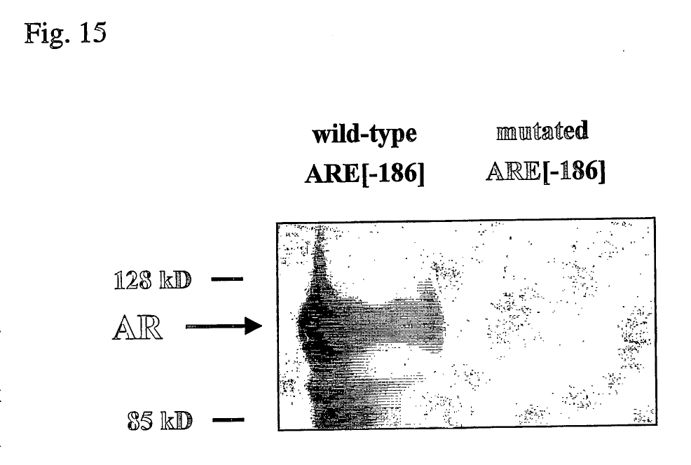

Addition of testosterone or synthetic testosterone, such as R1881, to the culture medium of prostate cancer cells, such as LNCaP, significantly upregulates TARP mRNA expression at the transcriptional level as illustrated by Northern blot in FIG. 9 and RT-PCR in FIG. 10. Furthermore, testosterone significantly increases the activity of both the proximal and large TARP promoters, as assessed by luciferase reporter gene assays on LNCaP cells, FIG. 12. The testosterone induction of TARP transcription is largely due to the presence of the ARE at position −186 in the TARP promoter. Upon testosterone or dihydro-testosterone binding to the cytosolic androgen receptor (AR), the AR is phosphorylated and forms a homodimer that is transported into the nucleus of a cell. In the nucleus, the homodimer activates transcription by binding to ARE in the promoter and enhancer sequences of target genes. Experimental data from luciferase reporter gene assays, FIG. 13, electrophoretic mobility shift assays, FIG. 14, and DNA precipitation assays, FIG. 15, presented herein demonstrate that the androgen receptor can bind the ARE in the proximal TARP promoter sequence and mediate testosterone induction.

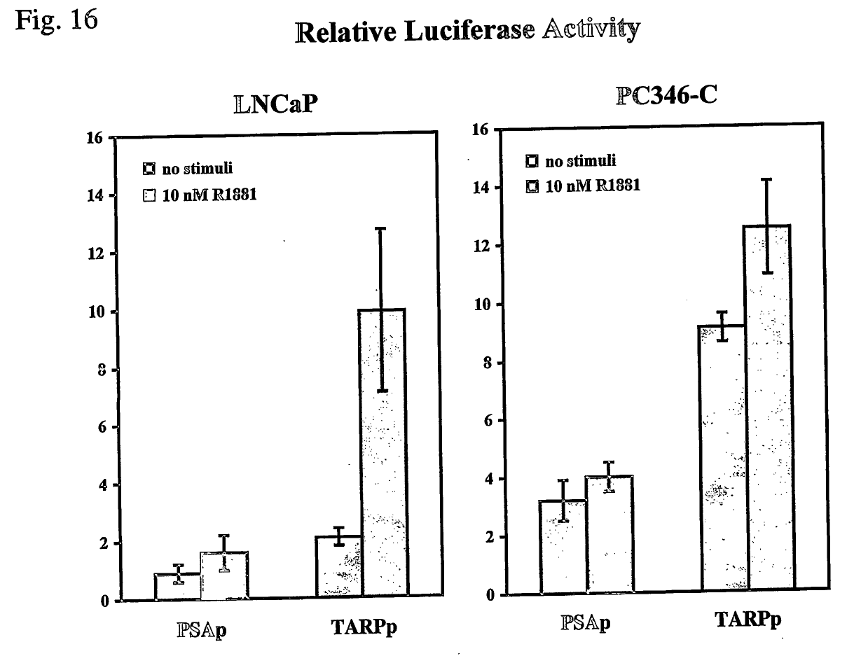

The promoter and enhancer sequences of the hKLK-3 gene that encodes the prostate specific antigen (PSA) have been one of the prior art regulatory sequences for expression of therapeutic molecules in prostate cancer cells. The regulatory sequences of PSA expression are the approximate 650 bp proximal PSA promoter at −632 to +12, in relation to the TIS of hKLK-3, and a 0.9 kb PSA enhancer at approximately −4750 to −3880, in relation to the TIS of hKLK-3. The PSA promoter and in particular the PSA enhancer is tightly regulated by androgens (Cleuijens et al., Mol. Endocrinol., 11: 148-161, 1997) although there are at least two regions of the PSA promoter that confer androgen-independent expression (Yeung et al., J. Biol. Chem. 275: 40846-40855, 2000). The PSA promoter has been extensively utilized for prostate-specific transgene expression (Gotoh et al., J. Urol., 160: 220-229, 1998; Latham et al., Cancer Res., 60: 334-341, 2000; Wu et al., Gene 77ter., 3: 1416-1426, 2001; Xie et al., Cancer Res., 61: 6795-6804, 2001). However, as illustrated in FIG. 16, the proximal TARP promoter of the invention yields a significantly higher reporter gene expression in the prostate cancer cell lines LNCaP and PC346-C than the proximal PSA promoter, especially when cells are cultured in the presence of testosterone (10 nM R1881). Thus, both the proximal TARP promoter and the large TARP promoter can be used for tissue specific gene expression in prostate cells, prostate cancer cells and female breast cancer cells with significantly higher activity than prior art employed promoters.

A recombinant regulatory sequence including the TARP promoter of the invention and other expression control sequences, e.g. enhancers, may be designed for increased activity. Such a regulatory sequence should preferably increase the activity of the TARP promoter and/or the inducibility thereof without significant loss in tissue specificity.

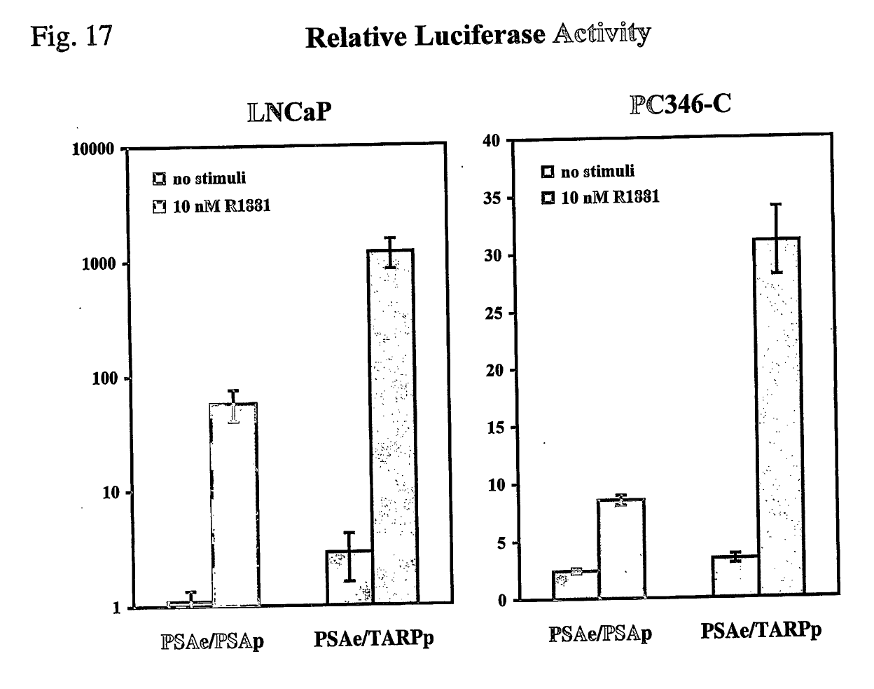

An embodiment of such a recombinant regulatory sequence comprises the PSA enhancer (PSAe) operatively linked to the proximal TARP promoter (TARPp). The sequence of the PSA enhancer is illustrated in FIG. 6. It includes an ARE, schematically illustrated in bold letters and underlined in FIG. 6. The transcriptional activity of the recombinant PSAe/TARPp regulatory sequence is approximately 20 times as strong as the PSAe/PSAp regulatory sequence in LNCaP cells and approximately 4 times as strong in PC346-C cells, when the cells are cultured in the presence testosterone (10 nM R1881), FIG. 17. The activity of the PSAe/PSAp and PSAe/TARPp regulatory sequences is strictly controlled by testosterone and therefore dependent on the presence of testosterone. Induction of transcription of TARP by testosterone is primarily regulated at the promoter level while induction of transcription of PSA by testosterone is primarily regulated at the enhancer level. This may explain the superior transcriptional activity of the recombinant PSAe/TARPp over the recombinant PSAe/PSAp regulatory sequence.

Another embodiment of a recombinant regulatory sequence according to the invention is to operatively link an enhancer of the prostate specific membrane antigen (PSMA) to a TARP promoter of the invention. PSMA expression is controlled by two characterized regulatory elements, the proximal 1.2 kb promoter upstream of the gene encoding PSMA (FOLH1) and the PSMA enhancer (PSMAe) located within the third intron of FOLH1. The PSMA promoter drives reporter gene expression but with less specificity than the PSA promoter (O'Keefe et al., Biochem. Biophys. Acta., 1443: 113-127, 1998) while the PSMA enhancer confers prostate-specific expression (Watt et al., Genomics, 73: 243-254, 2001). Furthermore, the activity of the PSMA enhancer is repressed by testosterone (Watt et al., Genomics, 73: 243-254, 2001). The nucleotide sequence of the PMSA enhancer is depicted in FIG. 7.

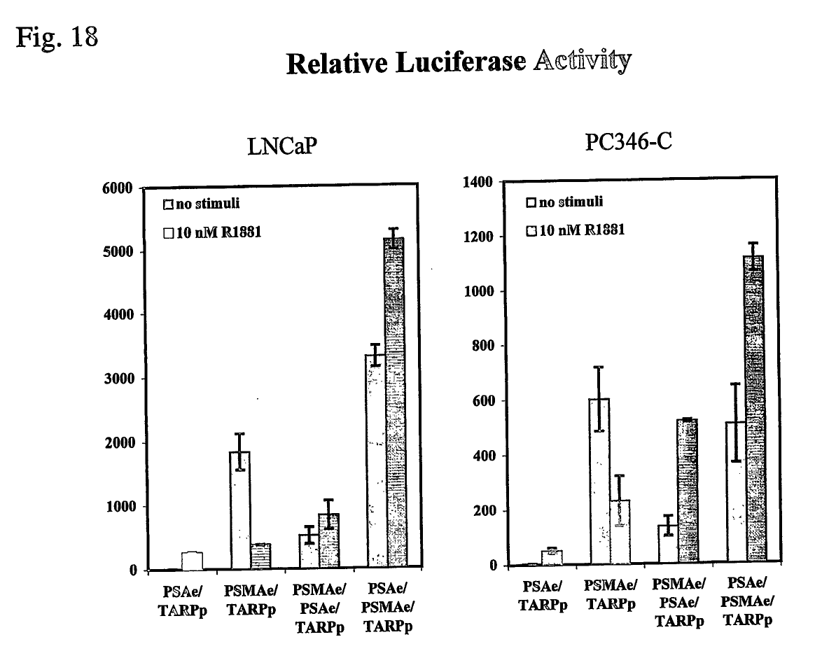

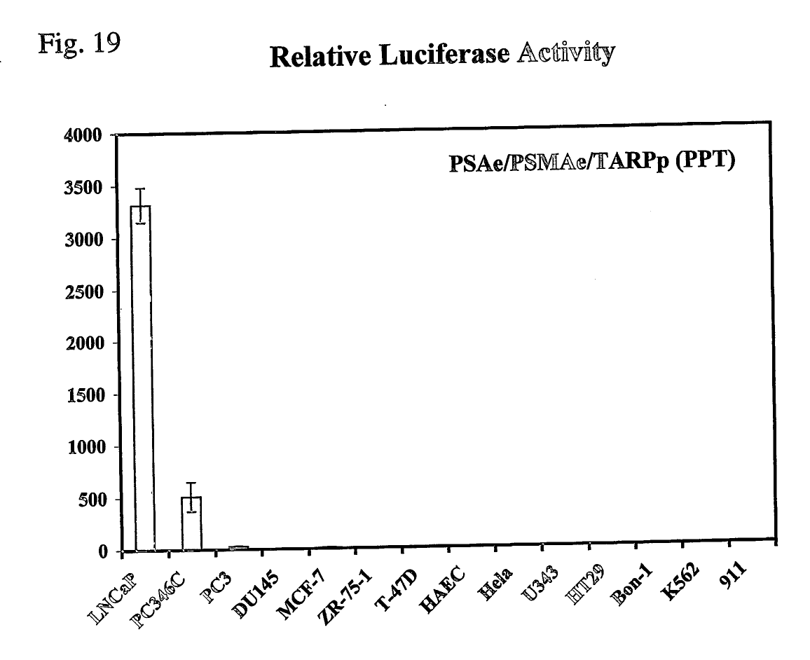

Operative linkage of the PSMA enhancer either to the TARP promoter alone or to the recombinant PSA enhancer/TARP promoter sequence, resulted in enhanced transcriptional activity in LNCaP and PC346-C, as illustrated in FIG. 18. The transcriptional activity of PSMAe/TARPp is higher in the absence than in the presence of testosterone, confirming the findings by Watt and colleagues that testosterone represses PSMA enhancer activity. The highest transcriptional activity in LNCaP and PC346-C is obtained with the recombinant regulatory sequence comprising the PSA enhancer and the PSMA enhancer operatively linked to the TARP promoter (PSAe/PSMAe/TARPp). The prostate-specific transcriptional activity of PSAe/PSMAe/TARPp was verified by luciferse reporter gene analysis on cell lines of various origins, FIG. 19. The fact that the recombinant PSMAe/TARPp, PSMAe/PSAe/TARPp and PSAe/PSMAe/TARPp regulatory sequences yield high reporter gene expression also in the absence of testosterone may be of importance in a future clinical setting of transcriptional targeting. Prostate cancer patients are often treated by androgen withdrawal. In these cases it may be beneficial to the patient to have a gene therapy vehicle harboring high prostate-specific gene expression that is not dependent on testosterone.

The present invention also comprises embodiments of linking the TARP promoter to two or more regulatory sequences, e.g. two enhancers. A typical example is when both the PMSA and PSA enhancers are operatively linked to the TARP promoter, e.g. the proximal TARP promoter, as mentioned above.

Other regulatory sequences for transgene expression in prostate cancer cells that can be operatively linked to the TARP promoter of the invention include, but are not restricted to, enhancer elements for transcriptional regulation of the kallikrein 2, kallikrein 4, osteocalcin, DD3, probasin and PSP94 genes. In addition, synthetic enhancers can be constructed using element for general factors required for the mechanics of initiating RNA synthesis, upstream factors to increase the efficiency of transcription initiation and response elements for inducible factors (reviewed by Nettelbeck et al., Trends Genet., 16: 174-181, 2000).

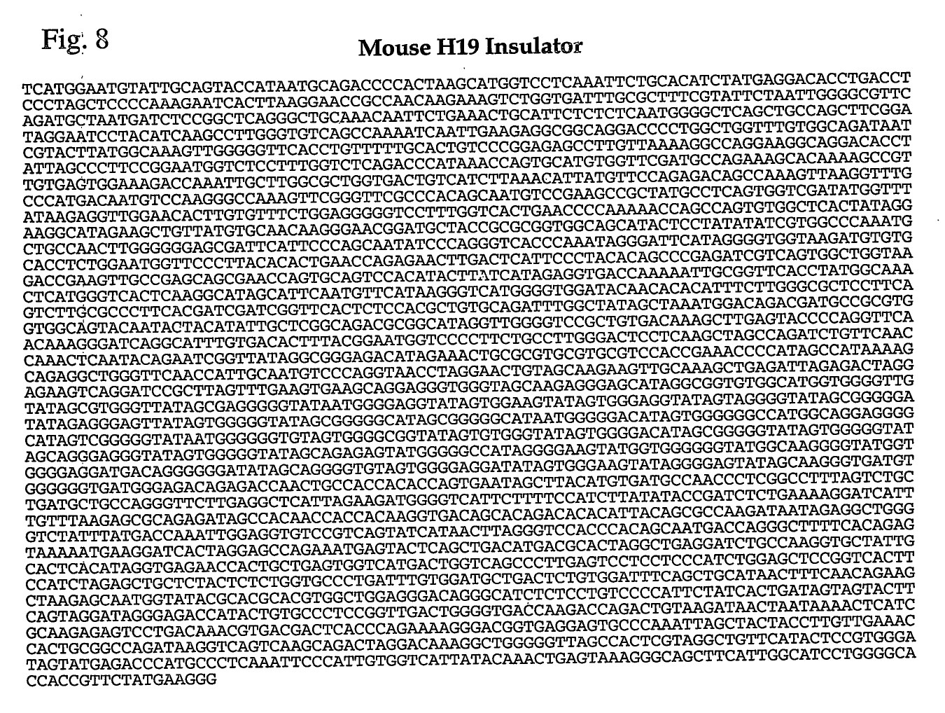

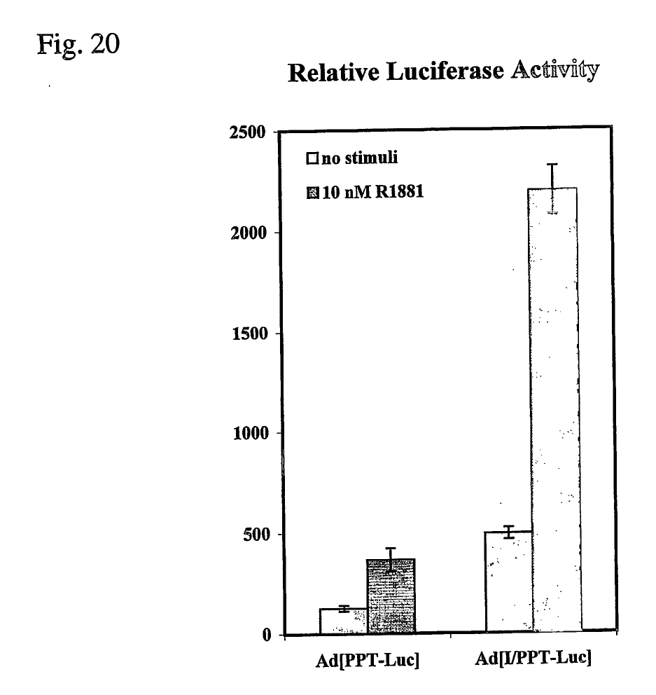

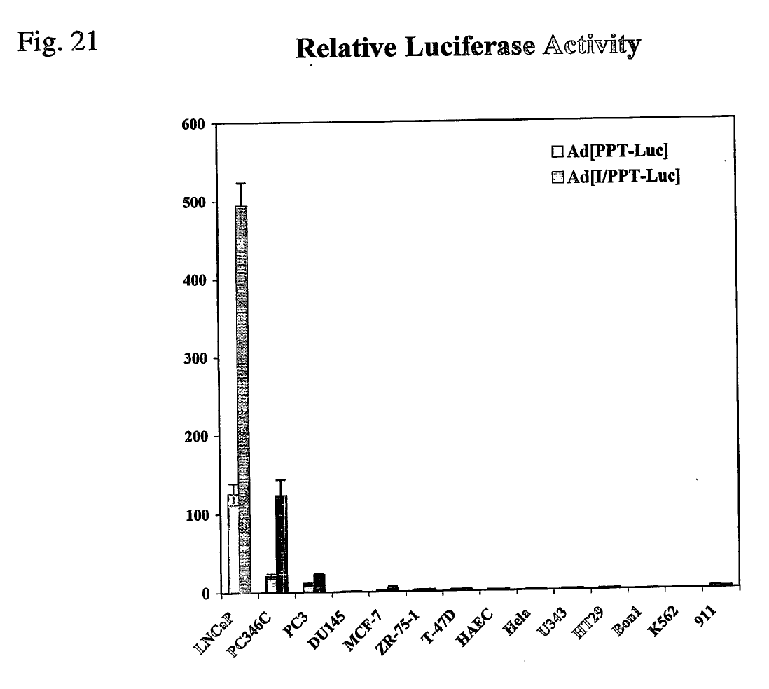

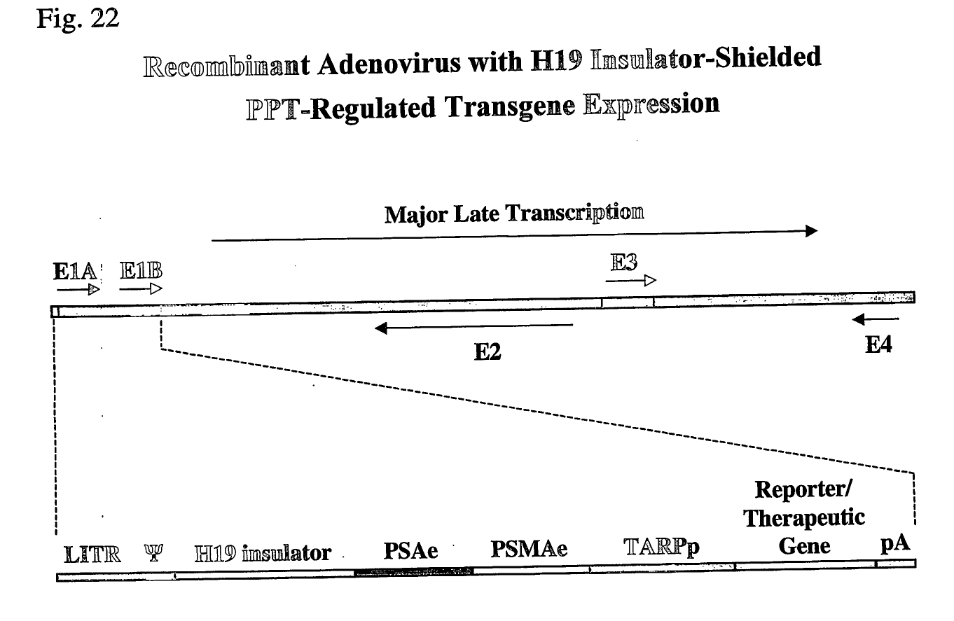

When a regulatory sequence of the invention, including the large or the proximal TARP promoter, either alone or linked to other expression control elements, e.g. the PSMA enhancer and the PSA enhancer, is introduced into an expression cassette of a viral vector to regulate expression of a transgene, the activity of the regulatory sequence may be interfered by viral sequences positioned upstream and/or downstream in the vector. This problem can be solved by shielding the regulatory sequence of the invention by inserting a DNA insulator in the vector upstream and/or downstream of the expression cassette. This DNA insulator will then physically separate the regulatory elements in the expression cassette from any interfering viral sequences, thereby lowering any risk for interference of activity. A typical DNA insulator that can be employed according to the present invention is the mouse H19 DNA insulator depicted in FIG. 8 (SEQ ID NO: 6). The H19 DNA insulator is located in the 5′ non-coding sequence of the H19 gene. When the H19 DNA insulator (1) is inserted in between the PSAe/PSMAe/TARPp regulatory sequence (PPT) and the upstream sequences comprising the left inverted terminal repeat (LITR) and the E1A enhancer, which overlaps with the encapsidation sequence (Ψ) needed for virus assembly, the transcriptional activity of the PPT sequence in prostate cancer cells is enhanced, FIG. 20. The prostate-specific transcriptional activity of the H19 insulator-shielded PPT sequence in a recombinant adenovirus serotype 5 vector was verified by analysis on cell lines of various origins, FIG. 21. An illustration of a recombinant adenovirus vector used is depicted in FIG. 22.

For high and specific therapeutic gene expression in breast cancer cells the TARP promoter is preferably combined with other expression control sequences, such as an enhancer for a gene encoding a breast-specific protein, which is preferably upregulated in response to estrogens.

According to an aspect of the invention there is provided the large and proximal TARP promoter, and a recombinant regulatory polynucleotide sequence comprising the large or the proximal TARP promoter together with other expression control elements, e.g. the PSA enhancer and the PSMA enhancer. In another aspect of the invention the regulatory sequence of the invention is operatively linked to a heterologous nucleotide sequence for providing tissue-specific expression thereof in prostate cells, prostate cancer cells and/or breast cancer cells.

Thus, a promoter according to the invention, i.e. the large or proximal TARP promoter, either alone or linked to other expression control elements, e.g. the PSA enhancer and/or the PSMA enhancer, may be operatively linked to a heterologous nucleic acid sequence for providing a tissue-specific and controllable expression thereof. The heterologous nucleic acid sequence may, preferably, code for a therapeutic molecule that, when expressed in a cancer cell, is capable of killing the cell, either directly or indirectly by sensitizing it to the effects of drugs or radiation or preventing or delaying division of the cancer cell. Strategies include but are not limited to tissue-specific: suicide gene therapy, proapoptotic molecule expression, toxin expression, radioisotopic therapy, immunomodulatory therapy, interference with the expression machinery of a protein essential for cell survival, and/or expression of a viral gene essential for viral replication, such as the adenoviral EIA gene. Therapeutic coding sequences can be placed under transcriptional control of constitutively active or tissue-specific promoters. The use of a sequence based on the TARP promoter will focus the activity of the therapeutic coding sequence to male prostate cells and prostate cancer cells and female breast cancer cells.

Description of Therapeutic Genes

The aim of suicide gene therapy is controlled cell death induction by converting a non-toxic prodrug into an active cytotoxic agent. Herpes simplex virus thymidine kinase (HSV-tk) in combination with ganciclovir (GCV) is one of the most investigated systems and in the context of prostate cancer it is the gold standard against which other gene directed enzyme prodrug therapy systems are compared. An adenovirus vector with HSV-tk under the transcriptional control of the osteocalcin promoter has been initiated in a clinical trial of prostate cancer, resulting in impressive responses in subcutaneous and bone sites with evidence of a local bystander effect mediated against bone stromal cells, which had the effect of depriving the prostate cancer cells of their essential stromal support (Koeneman et al., World J. Urol., 18: 102-110, 2000). E. coli cytosine deaminase (CD) catalyzes the conversion of non-toxic 5-fluorocytosine (5-FC) to toxic cytotoxic 5-fluorouracil (5-FU). O'Keefe et al. used the PSMA promoter and enhancer to direct expression of CD to prostate and prostate cancer cells (O'Keefe et al., Prostate, 45: 149-157, 2000). Adenovirus delivery of a fusion gene encoding CD and HSV-tk under control of a CMV promoter significantly increased the killing of prostate cancer cells (Blackburn et al., Cancer Res., 58: 1358-1362, 1998; Freytag et al., Gene Ther., 9: 1323-1333, 1998). Such viruses have been used in the clinics and show promising results when delivered intraprostatically in combination with external beam radiation therapy (Freytag et al., Cancer Res., 63: 7497-7506, 2003). Other suicide gene systems used include purine nucleoside phosphorylase/6-methylpurine, drosophilae melanogaster kinase/BVDU and nitroreductase/CB1954. The TARP promoter according to the present invention may preferably be operatively linked to one or several of the above identified, or other, suicide genes for providing an expression cassette in a vector, e.g. a recombinant adenoviral vector.

The cellular apoptotic machinery can be activated by the delivery of genes, which control elements for directing cells toward apoptosis. Proapoptotic gene products that have been utilized for prostate cancer gene therapy include caspase 1 (Shariat et al., Cancer Res., 61: 2562-2571, 2001), caspase 3 (Shariat et al., Cancer Res., 61: 2562-2571, 2001; Li et al., Cancer Res., 61: 186-191, 2001), caspase 7 (Li et al., Cancer Res., 61: 186-191, 2001), caspase 8 (Komata et al., Hum. Gene Ther., 13: 1015-1025, 2002), caspase 9 (Xie et al., Cancer Res., 61: 6795-6804, 2001) as well as Fas ligand, TRAIL and Bax (reviewed by Norris et al., Curr. Gene Ther., 1: 123-136, 2001). These and other apoptosis-inducing genes may be operatively linked to and consequently transcriptionally controlled by the TARP promoter of the invention.

Toxins are potent cytoreductive agents. Rodriguez et al. screened numerous bacterial toxins known to kill mammalian cells by cell cycle independent mechanisms to determine which would be best against prostate cancer (Rodriguez et al., Prostate, 34: 259-269, 1998). Diphtheria toxin was found to be the most toxic substance in their study. However, the toxin gene must be incorporated into a vector under tight regulatory control of a highly prostate specific promoter since diphtheria toxin is so toxic that even a small amount of leaky promoter activity in non-prostatic tissue may be lethal. The TARP promoter of the invention is a chief candidate for providing this highly prostate-specific promoter functionality and may therefore be linked to the gene coding for the diphtheria toxin.

The ability of normal and malignant thyroid cells to concentrate iodide is due to the sodium iodide symporter. Therefore, radioactive iodide has proven highly successful in the treatment of thyroid cancer. Spitzweg et al. proposed a novel form of gene therapy using prostate-specific regulatory sequences to direct expression of the sodium iodide symporter to prostate cancer cells followed by the delivery of radioactive iodide (Spitzweg et al., Cancer Res., 59: 2136-2141, 1999). The TARP promoter of the invention may be able to improve the usefulness of radioactive iodide therapy for prostate cancer treatment in male patients and extend the therapy to breast cancer treatment in female patients.

Tumor cells evade the immune system by decreasing their immunogenicity and dampening the effectiveness of immune responses mounted against them. Cytokines, such as interferons (IFNs), interleukins (ILs) and granulocyte-macrophage colony-stimulating factor (GM-CSF), can enhance immunogenicity and promote tumor regression. Therefore, introduction of such genes in tumor cells is an attractive approach of gene therapy. Most studies have been carried out using genes encoding GM-CSF and IL-2, although IL-4, IL-6, IL-7, IL-12, IFN-α, IFN-γ, and tumor necrosis factor (TNF)-α have been used as well (reviewed by Harrington et al., J Urol., 166: 1220-1233, 2001). Molecules for T cell co-stimulation such as B7.1/B7.2, CD154 and TRANCE may be used as well. A phase I clinical trial has been performed where prostate cancer patients received autologous irradiated tumor cells engineered to produce GM-CSF (Simons et al., Cancer Res., 59: 5160-5168, 1999). Furthermore, a phase I clinical trial has been performed in which 24 patients with locally advanced prostate cancer received IL-2 vaccine intraprostatically (Belldegrun et al., Hum. Gene Ther., 12: 883-892, 2001). Limited toxicity and evidence of systemic immune activation was observed. The TARP promoter of the present invention is suitable for gene-directed immunomodulatory therapy, by operatively linking it to a gene that enhances the immunogenicity of tumors and responsiveness of the immune system, e.g. one of the genes discussed above.

In many eukaryotes, expression of nuclear-encoded mRNA can be strongly inhibited by the presence of a double-stranded RNA corresponding to exon sequences in the mRNA. The process is known as RNA interference (RMAi) and is efficiently mediated by short double-stranded RNA molecules known as short interfering RNA (siRNA). These siRNA molecules can be expressed by the function of the cellular RNA polymerase III from gene promoters such as U6 and H1-RNA. The TARP promoter of the invention may be used to control the expression of a molecule encoding the H1-RNA promoter and the siRNA of a target gene in an expression cassette in a vector, e.g. recombinant virus vector. The protein encoded by the targeted gene is preferably vital for the cancer cells, whereby expression of the siRNA molecule due to the action of the TARP promoter kills or growth arrest the cancer.

Antisense molecules are DNA or RNA polynucleotides that are complementary to at least a portion of a specific mRNA molecule. In the cell, the antisense molecule hybridizes to the corresponding mRNA, forming a double stranded molecule. Such a double stranded RNA-RNA or DNA-RNA hybrids cannot be effectively translated and consequently the generation of its protein product will be diminished or prevented. The TARP promoter of the invention may be used to express an antisense molecule for specific inhibition of translation of a mRNA molecule which coding product is essential for prostate and/or breast cancer cells.

Ribozymes are RNA molecules that possess the ability of specific cleavage of other single-stranded RNA sequences. By modifying the nucleotide sequence of these ribozymes, it is possible to engineer them for recognizing and cleaving a specific mRNA sequence. Thus, a ribozyme may e.g. be designed to cleave an mRNA sequences coding for a protein that is essential for survival of a (prostate or breast) cancer cell (Raj and Liu, Gene, 313: 59-69, 2003). By operatively linking the TARP promoter of the invention to a ribozyme sequence it is possible to kill prostate and/or breast cancer cells, if the ribozyme cleaves and thereby prevents translation of a mRNA sequence that codes for a protein that is vital for the cells. In particular, ribozymes of the “hammerhead-type” recognizing a base sequence of 11 to 18 bases may be employed by the invention. The long recognition sequence of this type of ribozymes increases the likelihood that the sequence will occur exclusively in the target mRNA species.

By placing one or more viral genes important for viral replication, such as the adenoviral E1 genes, under transcriptional control of a prostate-specific regulatory sequence, viral vectors can be developed, which yields viruses that is replication competent in prostate and prostate cancer and breast cancer cells but attenuated in other tissues. Such adenoviral vectors are often referred to as conditionally replication competent adenoviruses (CRADs). The use of CRADs is a promising approach for cancer gene therapy because it allows efficient tumor penetration and spreading of the virus in a tissue-specific fashion. Such strategy has been demonstrated by insertion of human kallikrein 2, PSA and rat probasin promoters into human serotype 5 adenoviruses to specifically drive virus replication in prostate cancer cells Rodriguez et al., Cancer Res., 57: 2559-2563, 1997; Yu et al., Cancer Res., 59: 1498-1504, 1999; Yu et al., Cancer Res., 59: 4200-4203, 1999). These viruses show promising results in restricting virus replication and oncolytic activity to prostate and prostate cancer cells. Thus, since the TARP promoter is significantly more active than the PSA promoter but still is highly tissue-specific, the TARP promoter may successfully be introduced into a virus vector, e.g. human serotype 5 adenovirus, for prostate cell-, prostate cancer cell- and breast cancer cell-specific controlled replication of the oncolytic virus.

A replication-competent oncolytic adenovirus comprising one or more therapeutic genes, such as a suicide gene, a proapoptoic gene, a toxic gene and/or an immunomodulatory gene, have a great potential in prostate cancer gene therapy. A replication-competent adenovirus with a pair of therapeutic suicide genes, HSV-tk and CD, under transcriptional control of a CMV promoter has been initiated in the clinic and it shows promising results when delivered intraprostatically in combination with external beam radiation therapy (Freytag et al., Cancer Res., 63: 7497-7506, 2003). The TARP promoter of the invention may be used to restrict virus replication and/or therapeutic transgene expression to normal and neoplastic prostate epithelial cells. A CRAD whose replication is controlled by the TARP promoter of the invention may be constructed, that in addition includes one or more therapeutic genes, either controlled by a constitutively active promoter such as the CMV promoter or the TARP promoter of the invention. Such a virus may be delivered intraprostatically with potentially decreased risk of viral replication and therapeutic gene expression outside the prostate gland compared to an adenovirus whose replication is controlled by a constitutively active promoter such as the CMV promoter.

Description of Vector Systems

The development of vectors for gene therapy is an extremely active field of investigation (Somia and Verma, Nat. Rev. Genet., 1: 91-99, 2000) and over the past fifteen years, gene therapy has been moving from the laboratory to the clinic. The vectors allow introduction of a foreign gene into the cells of a subject. The foreign gene may be a therapeutic gene, as discussed above, e.g. for killing cancer cells. In such a case, it is vital that the therapeutic gene is only expressed in the targeted cells. One way to achieve this is to introduce the therapeutic gene into the vector under control of a regulatory sequence that is only active in the target cells. The TARP promoter and recombinant regulatory sequences comprising the TARP promoter are promising tissue-specific regulatory sequences, which may direct expression of therapeutic genes to prostate and breast cancer cells.

Injection of naked DNA, such as plasmids, into tumors represents the simplest system of gene delivery. A small percentage of the DNA is taken up and translocated to the cell nucleus, where it may be expressed transiently from an episomal location or stably if integration into the host genome occurs. To improve the efficiency of delivery the DNA is often complexed with cationic liposomes, or cationic polymers such as poly-L-lysine, polyethylenimine (PEI), polyglucos-amines and peptoids. For example, PEI has been shown to protect complexed DNA from degradation within endosomes and it also provides a means of promoting DNA release from the endosomal compartment and its subsequent translocation to the nucleus (Godbey et al., Proc. Natl. Acad. Sci. USA, 96: 5177-5181, 1999). Generally, these vectors cause little or no immunological reaction and are associated with limited toxicity in vivo. A liposome or polymer complex with the polynucleotide sequence of the invention, e.g. a plasmid, comprising the sequence of the TARP promoter, or a recombinant regulatory sequence including the TARP promoter, operatively linked to a heterologous coding sequence may be used according to the present invention.

Viral systems are by far the most effective means of DNA delivery, achieving high efficiencies of gene transfer, in many cases also in non-dividing cells. Virus vectors are attractive since viruses have evolved specific and efficient means to enter human cells and express their genetic material. The main challenge for viral vector development lies in keeping the targeting efficiency of viruses, while abrogating their ability to cause infection and disease. This is achieved by modifying the viral genome by removing sequences required for viral replication. The removed viral coding sequences can be replaced with an exogenous therapeutic gene. Such genetically engineered viruses theoretically retain wild-type viral cellular tropism and ensure transgene expression in the target cell population without causing ongoing infection (Kay et al., Nat. Med., 7: 33-40, 2001). To date there are four types of viral vectors in clinical trials: retroviruses, adenoviruses, herpes simplex virus and adeno-associated viruses. Other viruses that are under investigation include poxvirus, reovirus, Newcastle disease virus, alphavirus and vesicular stomatitis virus, which also may be employed as vectors according to the invention.

Adenoviruses are double strand DNA viruses of 30-35 kb in size. More than 40 adenovirus serotypes in 6 groups (A to F) have been identified. Adenovirus serotypes 2 (Ad2) and 5 (Ads), both belonging to group C, have been most extensively evaluated as candidates for gene delivery. Adenoviruses enter cells by binding to the coxsackie- and adenovirus receptor, which facilitates interaction of viral arginine-glycine-aspartate (RGD) sequences with cellular integrins. After internalization the virus escapes from cellular endosomes, partially disassembles and translocates to the nucleus, where viral gene expression begins. There are four different early genes expressed from the virus following infection (E1, E2, E3 and E4), encoding polypeptides important for regulating viral and cellular gene expression, viral replication, the inhibition of immune cell recognition and the inhibition of cellular apoptosis. Late in infection, the major late promoter is activated, resulting in the expression of polypeptides required for encapsidation of the virus. Adenoviruses can be converted for the use as vectors for gene transfer by deleting one or more of the early adenovirus genes E1 to E4 (reviewed by Russell, J. Gen. Virol., 81: 2573-2604, 2000). Deletion of E1 is especially useful since polypeptides from E1 are important for the induction of the E2, E3 and E4 promoters. Replication defective (E1-deleted) adenoviruses have a number of potential advantages as vectors for targeted gene delivery. They can be produced in high titers (10¹³ infectious virus particles per ml), they can infect both dividing and non-dividing cells and gene expression occurs without integration into the host genome, therefore minimizing the risk of insertional mutagenesis. An adenovirus vector comprising the sequence of the TARP promoter, or a recombinant regulatory sequence including the TARP promoter, operatively linked to a heterologous coding sequence may be used according to the present invention.

Recently, conditionally replication competent adenoviruses (CRAD) have been intensely investigated as gene delivery vehicles for cancer therapy (reviewed by Biederer et al., J. Mol. Med., 80: 163-175, 2002). Selective replication of an oncolytic virus in a tumor- or tissue-specific manner will, in theory, amplify the input dose and help spreading the toxic agent to adjacent tumor cells. This increases tumor transduction efficiency, creates a high local concentration of virus and thus augments therapeutic efficacy. CRAD, with conditional replication based on tissue-specific promoter-controlled E1A or E1A+E1B gene expression, yield viruses that specifically replicate in the cell type where the promoter is active. Such strategy has been demonstrated by insertion of human kallikrein 2, PSA and rat probasin expression control sequences into Ad5 to specifically drive virus replication in prostate cancer cells (Rodriguez et al., Cancer Res., 57: 2559-2563, 1997; Yu et al., Cancer Res., 59: 1498-1504, 1999; Yu et at., Cancer Res., 59: 4200-4203, 1999). The prostate-specific replicating adenoviruses (prostate CRAD) have entered clinical trials for prostate cancer treatment and show promising results. The TARP promoter of the invention is a very attractive regulatory sequence for tissue-specific expression of E1A or E1A+E1B for generation of a conditionally replicating oncolytic adenovirus that will specifically replicate in prostate, prostate cancer and breast cancer cells. Furthermore, the inclusion of an additional therapeutic gene in the TARP promoter-controlled CRAD may be employed according to the invention. The additional therapeutic gene may be but are not limited to: a suicide gene, a proapoptoic gene, a toxic gene or an immunomodulatory gene. It can be placed under transcriptional control of a constitutively active promoter, such as the CMV promoter, or a regulatory sequence comprising the TARP promoter of the invention.

The specificity and activity of tissue-specific promoters may be altered by adenoviral sequences in the vector backbone. Among the viral sequences that are potentially able to interfere with heterologous transgene promoters are the inverted terminal repeats (ITRs), the E1A enhancer, the E2, E4 and pIX promoters. All recombinant Ad5 and Ad2 vectors produced so far contain the adenoviral sequence from nucleotide 1 to 341 including the left ITR (LITR) and the E1A enhancer, which also contain the packaging sequence needed for virus assembly. Shi et al. demonstrated that a helper-dependent adenovirus lacking the E2, E4 and pIX promoters showed superior prostate-specific expression from the PSA promoter/enhancer as compared to traditional adenovirus (Shi et al., Hum. Gene Ther., 13: 211-224, 2002). However, interference on the promoter from the ITRs and the E1A enhancer may still result in loss of activity and/or loss of specificity. One approach to protect a transgene expression cassette comprising a tissue-specific promoter, such as the TARP promoter of the invention, from adenoviral backbone interference including the ITRs and the E1A enhancer, is to shield the transgene expression cassette with a DNA insulator, such as the mouse H19 DNA insulator discussed above, see FIG. 22.

According to an aspect of the invention there is provided a vector, e.g. a recombinant virus, comprising the nucleotide sequence of the TARP promoter or a transcriptionally active fragment thereof, either alone or operatively linked to other expression control sequences, such as the PSA enhancer and the PSMA enhancer. The TARP promoter-based regulatory sequence of the invention is operatively linked to a heterologous coding sequence for providing tissue-specific expression thereof.

An aspect of the invention is to provide a method of delivering a heterologous polynucleotide into a prostate cell, prostate cancer cell or breast cancer cell in vitro or in a subject (in vivo), preferably a human patient. The heterologous polynucleotide, operatively linked to a TARP promoter, or a transcriptionally active fragment thereof, or the TARP promoter together with other expression control sequences, is introduced into a cell, preferably by means of a vector, e.g. one of the vectors identified above.

Another aspect of the invention is to provide a method of expressing a heterologous polynucleotide in a prostate cell, prostate cancer cell or breast cancer cell in vitro or in a subject (in vivo) by introducing into a cell a TARP promoter, or a transcriptionally active fragment thereof, either alone or together with other expression control sequences, operatively linked to the heterologous polynucleotide. The expression cassette with the TARP promoter-based regulatory sequence and the operatively linked polynucleotide may be introduced into the cell by use of a vector. The vector is introduced into the cell under conditions that are effective in expressing the heterologous polynucleotide.

Furthermore, an aspect of the invention is to provide a method of treating and/or preventing a prostate-related disorder and/or a breast-related disorder, comprising introducing into a cell of a subject in need thereof, a TARP promoter, or a transcriptionally active fragment thereof, either alone or together with other expression control sequences, operatively linked to a heterologous polynucleotide coding for a molecule that is capable of killing the cell and/or preventing the disorder. The TARP promoter-based regulatory sequence and the operatively linked heterologous polynucleotide are preferably introduced into the cell by means of a vector.

Yet another aspect of the invention is use of the TARP promoter, or a transcriptionally active fragment thereof, either alone or together with other expression control sequences for the manufacturing of a medicament for the treatment of a prostate-related disorder, including prostate cancer, in males and a breast-related disorder, including breast cancer, in females. The treatment includes the therapeutic and/or prophylactic treatment of the disorder. The medicament then comprises a vector including the TARP promoter preferably operatively linked to a heterologous polynucleotide that codes for a molecule capable of preventing the disorder, e.g. by killing the cancer cells.

In therapeutic treatment the TARP-promoter-including vector is generally administered as a pharmaceutical formulation in admixture with a pharmaceutically acceptable adjuvant, diluent or carrier, which may be selected with due regard to the intended route of administration and standard pharmaceutical practice. The preferred administration route is via injection, preferably at the site of the disorder, e.g. delivered intraprostatically in the case of a prostate-related disorder.

### EXAMPLES

### Example 1

### Characterization of the TAE Promoter

The following studies present evidence that TARP mRNA is directly upregulated by testosterone at the transcriptional level. They further define the minimal TARP promoter required for transcriptional activity and they define an androgen response element (ARE) in the TARP promoter that is involved in testosterone induction of TARP mRNA transcription. The activity and specificity of the TARP promoter makes it a potential candidate for prostate cancer and breast cancer therapeutic gene expression.

TARP is Regulated by Testosterone at the Transcriptional Level

In order to determine whether TARP expression is regulated at the transcriptional level, Northern blot analyses and reverse transcription followed by polymerase chain reaction (RT-PCR) analyses were performed. The prostate adenocarcinoma cell line LNCaP was grown in steroid-depleted culture medium [RPMI-1640 (Invitrogen, Carlsbad, Calif.) supplemented with 5% charcoal/dextran-treated fetal bovine serum (FBS) (HyClone, Logan Utah), 2 mM L-glutamine (Invitrogen), 10 mM HEPES (Invitrogen) and 1 mM sodium-pyruvate (Invitrogen)] for 48 hours and then treated for 12 hours with 10 μM of the synthetic androgen, R1881 (NEN Life Science products, Boston, Mass.). Actinomycin-D (Act D, Sigma, St. Louis, Mo.) was added at 1 μg/ml to block RNA synthesis and cyclohexamide (CHX, Sigma) was added at 10 μg/ml to block protein synthesis. PolyA mRNA isolations and Northern blot hybridizations were performed as described in Essand et al. (Proc. Natl. Acad. Sci. USA, 96: 9287-9292, 1999). RT-PCR analysis was performed using conditions and primers described in Wolfgang et al. (Proc. Natl. Acad. Sci. USA, 97: 9437-9442, 2000, and Wolfgang et al., Cancer Res., 61: 8122-8126, 2001).

FIG. 9 illustrates the results from a Northern blot analysis of TARP mRNA level in LNCaP cells cultured in steroid-depleted medium without (−) and with (+) R1881, respectively. TARP mRNA level increased when the cells were cultured in medium supplemented with R1881. β-actin was used as mRNA control. The results of the RT-PCR analyses are illustrated in FIG. 10. It was confirmed that TARP mRNA level increased when LNCaP was cultured in medium supplemented with R1881. The increment in mRNA level caused by R1881 was completely abolished by the RNA synthesis inhibitor Act D, indicating that R1881 increases TARP expression through synthesis of new mRNA. The increase in mRNA expression caused by R1881 was not altered by the addition of the protein synthesis inhibitor CHX, indicating that R1881 acts directly on the synthesis of TARP mRNA, without requirement of newly synthesized second messenger proteins. Taken together, the results demonstrate that testosterone regulates TARP expression at the transcriptional level.

TARP Promoter Sequence Analysis

Genomic DNA was extracted from LNCaP cells using a DNA extraction kit (Qiagen, Valencia, Calif.). A 2700 bp genomic sequence, in between the Jγ1.1 and Jγ1.2 gene segments, was amplified by PCR using a proof-reading polymerase enzyme (Roche, Indianapolis, Ind.) and sequenced using BigDye terminator (Perlin-Elmer, Norwalk, Conn.). Additional sequence from the TCR γ locus was obtained from GenBank (AF159056). Nine thousand nucleotides of genomic DNA sequence, upstream of the transcription initiation site of TARP, was analyzed for regulatory sequence elements by using the MacVector 6.5 program (Oxford Molecular Group, Oxford, UK) with the addition of the ARE consensus sequence (Roche et al., Mol. Endocrinol., 6: 2229-2235, 1992), the NKX3.1 binding motif (Steadman et al., Nucleic Acids Res., 28: 2389-2395, 2000) and the PDEF binding motif (Oettgen et al., J. Biol. Chem., 275: 1216-1225, 2000).

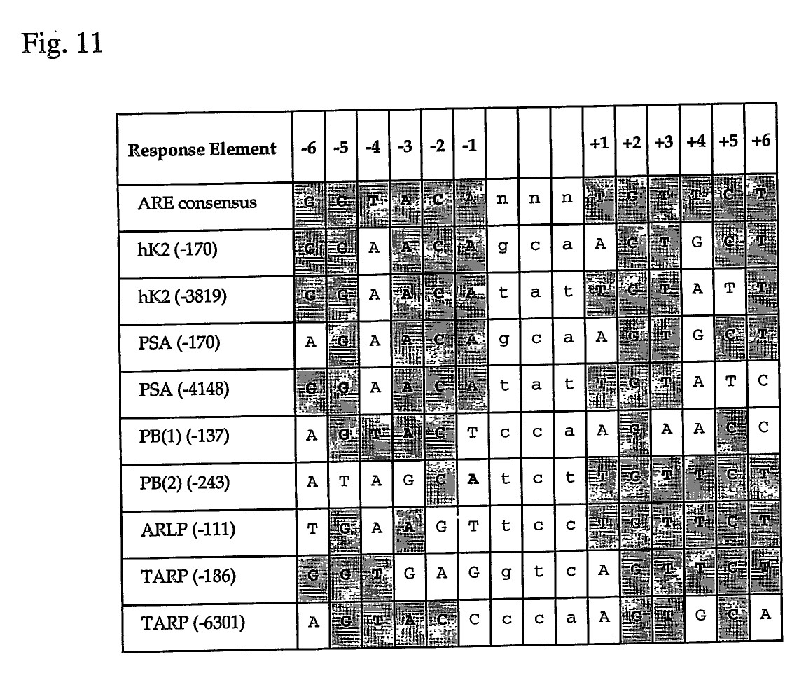

Several sequence element motifs with potential role in transcriptional regulation were identified upstream of the transcription initiation site (TIS) and are illustrated in the sequences of FIGS. 3, 4 and 5. These include a TATA-box (6/7 nucleotide matched sequence) located at −26, relative to the transcription start point (+1 in FIG. 3), a CAAT-box (8/9 nucleotide matched sequence) at −103 and three c-Jun binding sites at −111 (4/4 nucleotide matched sequence, anti-sense), −120 (4/4 nucleotide matched sequence) and −132 (4/4 nucleotide matched sequence). Furthermore, a potential transcription response region was identified at −1100, relative to the transcription start point, with a putative cAMP response element (CRE) (8/8 nucleotide matched sequence) and a putative activator protein-1 (AP-1) response element (7/7 nucleotide matched sequence), shown in the sequence of FIG. 4. Sequences with homology to the consensus ARE were found at several locations upstream of the transcription initiation site. FIG. 11 illustrates the homologies of putative AREs at −186, FIGS. 3 and 4, and −6301, FIG. 5, the ARE consensus sequence, and natural AREs in promoters and enhancers of androgen responsive genes, including human kallikrein 2 K2), prostate specific antigen (PSA), rat probasin (PB) and mouse aldose reductase-like protein (ARLP) genes. Nucleotides identical to the corresponding nucleotides in the ARE consensus sequence are presented in bold and highlighted.

The ARE consensus sequence consists of two asymmetric 6-bp half-site elements that are separated by a three-nucleotide-long spacer (Roche et al., Mol. Endocrinol., 6: 2229-2235, 1992). Natural AREs often have high homology to the consensus sequence on at least one half-site and the other half-site can deviate significantly. For example, the rat probasin element displays strong androgenicity in the context of its native promoter. It contains two AREs, both with high homology on one half-site and considerably less homology on the other half-site (Rennie et al., Mol. Endocrinol., 7: 23-36, 1993). The mouse aldose reductase-like protein (ARLP) has absolute homology with the ARE consensus sequence on the right half-site but only two nucleotides homology on the left half-site, yet it is a fully functional ARE (Fabre et al., J. Biol. Chem., 269: 5857-5864, 1994). The ARE at position −186 in the TARP promoter has high homology on the right half-site, 5/6 nucleotides match with the ARE consensus sequence while the left half-site diverges in positions −1, −2 and −3, FIG. 11. In contrast, AREs in the promoters and enhancers of hK2 (Yu et al., Cancer Res., 59: 1498-1504, 1999) and PSA (Schuur et al., J. Biol. Chem., 271: 7043-7051, 1996) have high over-all homology with the ARE consensus sequence, with complete match on positions ±2, ±3 and ±5 on both half-sites. Trapman and co-workers have shown strong testosterone-dependent induction of a reporter gene controlled by the minimal PSA promoter together with an upstream PSA enhancer (Cleutens et al., Mol. Endocrinol., 11: 148-161, 1997).

Characterization of the Proximal TARP Promoter

In order to identify the proximal TARP promoter, various portions of genomic DNA sequence from the 5′-flanking sequence of the TARP gene were analyzed.

Luciferase reporter constructs 5′-flanking sequences of the TARP gene were amplified by PCR from human genomic DNA (Roche) and MluI/XmaI-directionally cloned into the pGL3-Basic luciferase reporter gene plasmid (Promega, Madison, Wis.). The names given to reporter constructs indicate the first and last nucleotides of included genomic sequence, relative to the TARP transcription initiation site. The −6715 to −6233 sequence was KpnI/MluI-directionally cloned into T(−201, +45) to create T(−201, +45)+T(−6715, −6233). Inserted sequences were sequenced using BigDye terminator (Perkin-Elmer).

Transient Transfections and Luciferase Assay

LNCaP cells were transiently transfected by using Lipofectamine plus (Invitrogen) with reporter constructs described above together with a CMV/β-gal plasmid (Stratagene, La Jolla, Calif.) used as an internal control of transfection efficiency. The pGL3-Basic (Promega) was used for background luciferase activity. After transfection, cells were grown either in fresh normal culture medium [RPMI-1640 supplemented with 10% FBS, 2 mM L-glutamine, 10 mM HEPES and 1 mM sodium-pyruvate] or in fresh steroid-depleted culture medium [RPMI-1640 supplemented with 5% charcoal/dextran-treated fetal bovine serum, 2 mM L-glutamine, 10 mM HEPES and 1 mM sodium-pyruvate] in the presence or absence of 10 nM of R1881. After 36 hours cells were lysed with lysis buffer (PharMingen, San Diego, Calif.). Luciferase activities were determined in duplicate samples as suggested by the manufacturer (PharMingen). Luciferase activities were calculated by dividing the relative light unit value with the β-galactosidase value.

FIG. 12 illustrates the relative luciferase activity of reporter constructs containing different portions of the 5′ flanking sequence of TARP in LNCaP cells grown in normal culture medium. Average activities with standard deviation from three independent experiments with duplicate samples are shown. The T(−201, +45) construct yielded a luciferase activity of 7 times above background level (pGL3-Basic), and shorter constructs yielded activities of 2 times or less above background level. The highest activity was observed for T(−2646, +45) at approximately 18 times above background, and the activity for T(−6767, +45) was significantly lower, indicating the presence of a silencing region upstream of −2646. No significant difference in activity between T(−1042, +45) and T(−1168, +45) was observed, even though the latter construct contains putative CRE- and AP-1-binding sites.

In order to investigate the regulatory role of testosterone on TARP transcription, LNCaP cells were transiently transfected with reporter constructs and grown in steroid-depleted culture medium with or without 10 nM of R1881 before analysis of luciferase activity, FIG. 13. Average activities with standard deviation from three independent experiments with duplicate samples are shown. The synthetic androgen caused a 4-fold induction of activity for T(−201, +45), but no induction was observed for T(−181, +45), indicating the presence of a functional ARE in T(−201, +45) that is not present in T(−181, +45). Therefore, the ARE-like sequence at −186, relative to the transcription initiation site, appears to be important for testosterone-dependent expression of TARP. The T(−6767, +45) construct yielded only 2-fold induction, further indicating the presence of a silencing region upstream of −2646. A reporter construct with the T(−201, +45) sequence and an upstream region (−6715 to −6233) containing a putative ARE at −6301 yielded slightly higher induction by testosterone than T(−201, +45). Taken together, the ARE at −186 appear to be essential for testosterone-dependent transcriptional regulation of TARP, but the ARE-like sequence at −6301 appear to be of lesser importance.

Characterization of the ARE in the Proximal TARP Promoter

In order to determine whether the androgen receptor (AR) is able to bind the ARE sequence at −186 electrophoretic mobility shift assays (EMSA) and DNA precipitation (DNAP) assays were performed using in vitro-translated AR and total protein extract of LNCaP cells, respectively.

Electrophoretic Mobility Shift Assay (EMSA)

Double-stranded oligonucleotides constituting either the wild-type ARE or a mutated version thereof at −186, relative to the TARP transcription initiation site, were 5′end labeled with [γ³²P]dATP using T4 polynucleotide kinase (Amersham Bioscience, Piscataway, N.J.). The wild-type oligonucleotide sequence was GCAAGGTGAGGTCAGTTCTTAAA and includes the ARE[−186] sequence of the TARP promoter. The corresponding oligonucleotide sequence with the mutated version of the ARE[−186] was GCAAGGTGTGGTCAACTATTAAA. Full-length human AR cDNA was subcloned from PSVAR0 (Brinkmann et al., J. Steroid Biochem., 34: 307-310, 1989) to pBluescript II SK+ (Stratagene). In vitro-translated human AR was produced by transcription-coupled translation by using T7 RNA polymerase and wheat germ extract (Promega). Translated AR was incubated with either wild-type ARE[−186] probe or mutated ARE[−186] probe in a buffer containing 20 mM HEPES (pH 7.5), 30 mM KCl, 2 mM MgCl₂, 1 mM EDTA, 1 mM dithioerythritol, 1 mM phenylmethylsulfonyl fluoride, 10 nM R1881, 15% glycerol, 1 μg poly(dI-dC)poly(dI-dC) and 1 μg BSA. Samples were resolved by electrophoresis on 5% TBE/polyacrylamide gels. The gels were dried and subjected to autoradiography.

FIG. 14 illustrates a resulting gel from the EMSA showing a complex between the in vitro-translated AR and the ³²P-labeled ARE[−186] probe. This DNA-protein complex is specific for ARE[−186] (FIG. 14, lane 2), because formation of this complex was completely abolished by a 100-fold molar excess of non-labeled ARE[−186] competitor (FIG. 14, lane 3). Furthermore, in vitro-translated AR was not able to bind a mutated ARE sequence because the specific DNA-protein complex observed with the wild-type ARE[−186] probe (FIG. 14, lane 5) was not detected with the mutated ARE[−186] probe (FIG. 14, lane 7).

DNA Precipitation (DNAP) Assay

Double-stranded, biotinylated oligonucleotides were prepared for wild-type ARE[−186] and mutated ARE[−186], as described above for EMSA. LNCaP total extract was prepared in radioimmunoprecipitation assay (RIPA) buffer [10 mM Tris (pH 7.5), 1 mM EDTA, 150 mM NaCl, 1% Triton X-100, 0.5% deoxycholic acid, 0.1% SDS, 1 mM Pefabloc SC (Roche)]. Total extract was precleared with streptavidin-agarose (Amersham Biosciences) and incubated with biotinylated probes for 1 hour at 4° C. DNA-bound proteins were precipitated with streptavidin-agarose for 30 minutes at 4° C., washed three times with RIPA buffer, resolved by 4-20% SDS-PAGE (BioRad, Hercules, Calif.) and detected by Western blot analysis using a mouse monoclonal anti-AR antibody (AR441, Santa Cruz Biotechnology, Santa Cruz, Calif.).

FIG. 15 illustrates the results from the DNAP assay. It demonstrates that the wild-type ARE[−186] oligonucleotide was able to precipitate AR from the total extract of LNCAP, as detected by Western blot using an anti-AR antibody. On the contrary, the mutated ARE[−186] oligonucleotide was not able to precipitate AR from the total extract.

Thus, the proximal TARP promoter comprises a functional ARE at −186, relative to the TRAP transcription initiation site, that is able to bind the androgen receptor. The native ARE at −186 in the TARP promoter appears to be potent in testosterone induction of TARP mRNA expression.

### Example 2

### Recombinant Regulatory Sequences for Prostate-Specific Expression

The following studies present evidence that the TARP promoter can be combined with heterologous enhancer elements for increased transcriptional activity in prostate cancer cells without compromising the tissue specificity. The highest activity is obtained for a recombinant regulatory sequence composed of the TARP promoter operably associated with the PSMA enhancer and the PSA enhancer (PSAe/PSMAe/TARPp). Importantly, this regulatory sequence is transcriptionally active in prostate cancer cell lines, grown in culture medium either with or without the addition of testosterone, while transcriptional activity is not observed in other cell lines. A recombinant adenovirus with the PSAe/PSMAe/TARPp regulatory sequence, possibly shielded from upstream adenoviral sequences by the mouse H19 insulator, provides specific transgene expression in prostate cancer cell lines. Thus, the studied recombinant regulatory sequences can be used to restrict expression of therapeutic genes to prostate cancer cells and may therefore play a role in prostate cancer gene therapy.

Cell Lines

The prostate adenocarcinomas LNCaP, PC346-C, PC-3 and DU145; the breast adenocarcinomas MCF 7 and ZR-75-1; the colon carcinoma HT-29; the glioma U343; and the chronic myeloid leukemia K562 were cultured in RPMI-1640 supplemented with 10% FBS, 2 mM L-glutamine, 10 mM HEPES and 1 mM sodium-pyruvate. The cervix adenocarcinoma HeLa; the embryonic retinoblast 911; and the breast adenocarcinoma T47D were cultured in DMEM supplemented with 5% FBS and 2 mM L-glutamine. The pancreatic carcinoid Bon-1 was cultured in DMEM:F12K Nutrient Mixture at a 1:1 ratio, supplemented with 10% FBS, 2 mM L-glutamine and 1 mM sodium-pyruvate. The aortic endothelial HAEC was cultured in Medium 200 (Cascade Biologics, Portland, Oreg.) supplemented with the low serum growth supplement kit (Cascade Biologics). Steroid-depleted cell culture medium was RPMI-1640 supplemented with 5% charcoal/dextran-treated FBS, 2 mM of L-glutamine, 10 mM of HEPES and 1 mM of sodium-pyruvate.

Characterization of Prostate-Specific Recombinant Regulatory Sequences

In order to develop a potent and specific transcriptional regulatory sequence for gene expression in prostate and prostate cancer cells the following regulatory sequences were studied:

1) TARP promoter (TARPp);

2) PSA promoter (PSAp);

3) PSA enhancer operatively linked to PSA promoter (PSAe/PSAp);

4) PSA enhancer operatively linked to TARP promoter (PSAe/TARPp);

5) PSMA enhancer operatively linked to TARP promoter (PSMAe/TARPp);

6) PSMA enhancer and PSA enhancer operatively linked to TARPp (PSMAe/PSAe/TARPp);

7) PSA enhancer and PSMA enhancer operatively linked to TARPp (PSAe/PSMAe/TARPp); and

8) H19 insulator (I) shielding the PSAe/PSMAe/TARPp sequence (I/PSAe/PSMAe/TARPp).

Luciferase Reporter Constructs

The proximal TARP promoter construct (TARPp) was generated by PCR amplification of the TARP promoter sequence from nucleotide −201 to +45, FIG. 3, followed by insertion into pGL3-Basic (Promega). The PSA73luc plasmid (Cleutens et al., Mol. Endocrinol., 11: 148-161, 1997), containing the PSA promoter (PSAp), from −632 to +12, the PSA enhancer (PSAe), from −4758 to −3884, FIG. 6, and a luciferase reporter gene was a kind gift from Dr. J. Trapman, Erasmus University, Rotterdam, the Netherlands. For comparison reasons the PSA73luc plasmid is designated PSAe/PSAp in the present description. The proximal PSA promoter construct was generated by removal of the PSA enhancer from PSAe/PSAp. The recombinant PSAe/TARPp construct was generated by insertion of the PSA enhancer upstream of the TARP promoter sequence in TARPp. The PSMA enhancer (PSMAe) sequence from nucleotide +12272 to +12530, FIG. 7, was amplified by PCR from human genomic DNA (Roche) and inserted upstream of the TARP promoter sequence in TARPp to generate the PSMAe/TARPp construct. The PSMAe/PSAe/TARPp construct was generated by insertion of PSMAe in PSAe/TARPp and the PSAe/PSMAe/TARPp construct was generated by insertion of PSAe in PSMAe/TARPp. Inserted sequences were sequenced (BigDye terminator; Perkin-Elmer).

Transient Transfections and Luciferase Assay

Cells were transiently transfected by using Lipofectamine plus (Life Technologies) with reporter constructs described above together with a CMV/β-gal plasmid (Stratagene) used as an internal control of transfection efficiency. pGL3-Basic (Promega) was used for background luciferase activity. After transfection, cells were grown in fresh steroid-depleted culture medium with or without 10 nM of R1881. After 36 hours cells were lysed with lysis buffer (PharMingen). Luciferase activities were determined in duplicate samples as suggested by the manufacturer (PharMingen). Luciferase activities were calculated by dividing the relative light unit value with the β-galactosidase value.

FIG. 16 illustrates the transcriptional activity of the proximal PSA promoter and the proximal TARP promoter in LNCaP and PC346-C cells cultured either under steroid-depleted conditions (no stimuli) or under testosterone-rich conditions (10 nM R1881). Average activities, expressed in relation to pGL3-Basic, from three independent experiments with duplicate samples are shown. No significant activity for the PSA promoter was detected in LNCaP cells neither with nor without addition of testosterone. Under steroid-depleted conditions (no stimuli), the TARP promoter yielded a luciferase activity of 2.1 times above background (pGL3-Basic) and with testosterone (10 nM R1881) the relative activity was 9.9 above background. The transcriptional activity of the TARP promoter was higher than the activity of the PSA promoter also in PC346-C cells. FIG. 17 illustrates the transcriptional activity of a construct containing the PSA enhancer and PSA promoter (PSAe/PSAp) and a construct containing the PSA enhancer and TARP promoter (PSAe/TARPp) in LNCaP and PC346-C cells. The PSAe/PSAp showed very low basic transcriptional activity in LNCaP under steroid-depleted conditions (no stimuli), although when cells were grown with testosterone (10 nM R1881), the transcriptional activity was 58 times above background. The recombinant PSAe/TARPp showed a transcriptional activity of 2.9 above background under steroid-depleted conditions (no stimuli) and an activity of 1188 above background in the presence of testosterone (10 nM R1881). Therefore, the induction of the PSAe/TARPp caused by testosterone is 400-fold, and under those conditions the transcriptional activity of PSAe/TARPp is 20 times higher than the activity of the PSAe/PSAp. Because LNCaP expresses a mutated androgen receptor we also examined the activities in PC346-C, prostate cancer cell line expressing a normal androgen receptor. The transcriptional activity of PSAe/TARPp was higher than the activity of PSAe/PSAp also in PC346-C cells, although the differences were less pronounced than in LNCaP cells.

In order to obtain regulatory sequences with testosterone-independent but prostate-specific expression the PSMA enhancer was included. The PSAe/TARPp, PSMAe/TARPp, PSMAe/PSAe/TARPp and PSAe/PSMAe/TARPp constructs were analyzed side by side in transiently transfected LNCaP and PC346-C cells cultured either under steroid-depleted conditions (no stimuli) or under testosterone-rich conditions (10 nM R1881). FIG. 18 illustrates the average transcriptional activities of the various regulatory sequences, expressed in relation to pGL3-Basic, from three independent experiments with duplicate samples. The PSMAe/TARPp construct yielded a transcriptional activity approximately 1800 times above background in LNCaP without testosterone (no stimuli). The transcriptional activity was repressed 5-fold by testosterone (10 nM R1881) to 350 times above background. The PSMAe/PSAe/TARPp reporter construct showed a 1.5-fold lower basal activity under steroid-depleted conditions than with testosterone added to the culture medium. The PSAe/PSMAe/TARPp construct yielded the highest activities of approximately 3300 times above background under steroid-depleted conditions (no stimuli) and approximately 5150 times above background with testosterone (10 nM R1881). The activities were lower in PC346-C than in LNCaP cells but followed the same pattern of induction and repression in response to testosterone.

The prostate specificity of the PSAe/PSMAe/TARPp (PPT) transcription regulatory sequence was confirmed in luciferase reporter gene assays by screening cell lines of different origins. FIG. 19 illustrates the relative transcriptional activity of PPT under steroid-depleted conditions. Average activities, expressed in relation to pGL3-Basic, from three independent experiments with duplicate samples are shown. Activity was only observed in the prostate cancer cell lines LNCaP, PC346-C and PC3, demonstrating prostate-specificity of the PPT regulatory sequence.

Construction of Recombinant Adenoviruses for Specific Transgene Expression in Prostate Cells Both in the Absence and Presence of Testosterone

Production of Recombinant Adenovirus

Replication-deficient adenoviruses were produced using the AdEasy system (He et al., Proc. Natl. Acad. Sci. USA., 95: 2509-2514, 1998), as suggested by the manufacturer (Stratagene). The different expression cassettes, with a regulatory sequence and a luciferase reporter gene, as described above, were XhoI/SalI-directionally subcloned from the pGL3-Basic environment to pShuttle (a kind gift from Dr. B Vogelstein, Johns Hopkins, Baltimore, Md.). The H19 insulator, FIG. 8., nucleotide sequence from −3409 to −284 relative to the mouse H19 transcription start point, was excised from pGEM-ICR (a kind gift from Dr. R Ohlsson, Uppsala University, Uppsala, Sweden), treated with Klenow to produce blunt ends and inserted in the EcoRV site of pBluescript II SK (Stratagene). The H19 insulator was then subcloned in the sense direction, using KpnI/NotI, from pBluescript II SK into a pShuttle vector containing the PSAe/PSMAe/TARPp-luc expression cassette. A PSAe/TARPp fragment was excised from the pGL3-Basic environment, treated with Klenow to produce blunt ends and inserted in the EcoRV site of a pShuttle vector containing the H19 insulator and a luciferase reporter gene to generate the negative control virus Ad[Antisense-Luc]. The reverse orientation of PSAe/TARPp was confirmed by restriction digest. A GFP expressing adenovirus Ad[CMV-GFP] was generated from pAdTrack-CMV (a kind gift from Dr. B Vogelstein, Johns Hopkins, Baltimore, Md.). Viruses were produced in 911 cells (Crucell, Leiden, the Netherlands) and viral titers were determined using a standardized plaque assay.

Viral Transduction and Luciferase Assays

Cells were transduced at a multiplicity of infection (MOI) yielding 50-90% transduced cells, as determined by transduction of each cell line with Ad[CMV-GFP] and assessing the percentage of GFP-expressing cells by flow cytometry. The MOI for 911 cells was 1; for PC346-C, HAEC, Bon-1, U343 it was 10; for LNCaP, HeLa, PC3, ZR75-1, T47D it was 50; and for DU145, HT29, K562, MCF-7 it was 100. Luciferase activities were determined in triplicate samples as suggested by the manufacturer (PharMingen). The relative luciferase activities were expressed in relation to background activity of Ad[Antisense-Luc].

By comparing the activity of the PSAe/PSMAe/TARPp regulatory sequence (PPT) with the activity of a constitutively active CMV promoter in LNCaP cells, we found that they were equally strong in the pGL3-Basic environment. However, when placed in the E1 position, forward direction of a recombinant adenovirus serotype 5 vector (Ad), the PPT activity was only approximately 10% of the CMV promoter activity placed in the same position. By introducing the mouse H19 insulator (I) between the PPT sequence and the upstream adenoviral sequences the transcriptional activity of the PPT regulatory sequence is enhanced to approximately 50% of the CMV promoter activity. The adenoviral sequences, upstream of the expression cassette, comprise the left inverted terminal repeat (LITR) and the encapsidation sequence (Ψ), which overlaps the E1A enhancer, see FIG. 22. These sequences could potentially interfere with the PPT sequence in the expression cassette.

FIG. 20 illustrates a comparison between two adenoviruses with the PPT-luc transgene expression cassettes. The Ad[I/PPT-Luc] has the H19 insulator upstream of the regulatory sequence, to shield it from interference of the LITR/Ψ(E1A enhancer) sequence, while Ad[PPT-Luc] does not have an insulator. The Ad[PPT-Luc] yielded a transcriptional activity about 120 times above background (Ad[Antisense-Luc]) under steroid-depleted conditions (no stimuli) and an activity of 360 times above background with testosterone (10 nM R1881). The Ad[I/PPT-Luc] yielded a transcriptional activity about 500 times above background under steroid-depleted conditions (no stimuli) and an activity of 2200 times above background with testosterone (10 nM R1881). Therefore, the transcriptional activity of Ad[I/PPT-Luc] is more than 4-fold higher (without testosterone) and 6-fold higher (with testosterone) than the activity of Ad[PPT-Luc].

The prostate cell-specific transcriptional activity of Ad[PPT-Luc] and Ad[I/PPT-Luc] were analyzed on cell lines of various origins, cultured under steroid-depleted conditions, FIG. 21. Transcriptional activity was only observed in the prostate cancer cell lines LNCaP, PC346-C and PC3, demonstrating prostate specificity of the Ad[PPT-Luc] and Ad[I/PPT-Luc] adenoviruses.

Thus, the PPT regulatory sequence provides prostate-specific activity and it is active in prostate cancer cells cultured either with or without testosterone in the culture medium. Since prostate cancer patients are often treated by androgen withdrawal, it may be beneficial to the patient to have a gene therapy vehicle harboring high prostate-specific gene expression that is not dependent on testosterone.

It will be understood by a person skilled in the art that various modifications and changes may be made to the present invention without departure from the scope thereof, which is defined by the appended claims.

1-24. (canceled)

25. A method of providing expression of a molecule in a prostate cell, prostate cancer cell or breast cancer cell, comprising introducing into said cell a TARP promoter, or a transcriptionally active fragment thereof, operatively linked to a heterologous polynucleotide that codes for said molecule, under conditions effective to direct expression of said molecule in said cell.

26. The method according to claim 25, wherein said heterologous polynucleotide codes for a therapeutic molecule with the capacity of directly or indirectly killing said cell.

27. The method according to claim 25, wherein said TARP promoter is operatively linked to a heterologous enhancer sequence selected from at least one of:
PSA enhancer;
PSMA enhancer;
PSA enhancer operatively linked to the PSMA enhancer; and
PSMA enhancer operatively linked to the PSA enhancer.

28. A method of treating and preventing a prostate-related or breast-related disorder, comprising introducing into a cell of a subject in need thereof, a TARP promoter, or a transcriptionally active fragment thereof, operatively linked to a heterologous polynucleotide that codes for a molecule capable of killing said cell or capable of preventing said disorder.

29. The method according to claim 28, wherein said TARP promoter is operatively linked to a heterologous enhancer sequence selected from at least one of:
PSA enhancer;
PSMA enhancer;
PSA enhancer operatively linked to the PSMA enhancer; and
PSMA enhancer operatively linked to the PSA enhancer.

30. An isolated polynucleotide sequence comprising a TARP promoter, or a transcriptionally active fragment thereof, operatively linked to a heterologous expressible nucleotide sequence.

31. The sequence according to claim 30, wherein said TARP promoter is operatively linked to a heterologous enhancer sequence selected from at least one of:
PSA enhancer;
PSMA enhancer;
PSA enhancer operatively linked to the PSMA enhancer; and
PSMA enhancer operatively linked to the PSA enhancer.

32. The sequence according to claim 30, wherein said TARP promoter has a nucleotide sequence selected from at least one of:
nucleotide 1 to 201 of a nucleotide sequence of SEQ ID NO: 1;
nucleotide sequence of SEQ ID NO: 1; and
nucleotide 1 to 2646 of a nucleotide sequence of SEQ ID NO: 2.

33. An isolated polynucleotide sequence comprising a TARP promoter, or a transcriptionally active fragment thereof, operatively linked to other expression control sequences.

34. The sequence according to claim 33, wherein said TARP promoter is operatively linked to a heterologous enhancer sequence selected from at least one of:
PSA enhancer;
PSMA enhancer;
PSA enhancer operatively linked to the PSMA enhancer; and
PSMA enhancer operatively linked to the PSA enhancer.

35. The sequence according to claim 33, wherein said TARP promoter has a nucleotide sequence selected from at least one of:
nucleotide 1 to 201 of a nucleotide sequence of SEQ ID NO: 1;
nucleotide sequence of SEQ ID NO: 1; and
nucleotide 1 to 2646 of a nucleotide sequence of SEQ ID NO: 2.

36. An expression cassette comprising the polynucleotide sequence of claim 30.

37. A vector comprising the expression cassette of claim 36.

38. An expression cassette comprising the polynucleotide sequence of claim 33.

39. A vector comprising the expression cassette of claim 38.


SEQUENCE LISTING (18 sequences)

SEQ ID NO: 1 - DNA, 246 bp, Homo sapiens
  CAAT_signal (99)..(107)
  TATA_signal (176)..(182)
  misc_signal (16)..(30): Androgen Response Element (ARE)
  protein_bind (70)..(73): c-Jun binding site
  protein_bind (82)..(85): c-Jun binding site
  protein_bind (91)..(94): c-Jun binding site
  ref <308>: DATABASE ACCESSION NUMBER: GenBank / AF151104
  ref <309>: DATABASE ENTRY DATE: 1999-08-23
  ref <313>: RELEVANT RESIDUES: (2476)..(2721)
aagtgcccagggcaaggtgaggtcagttcttaaattcctgattacattcagatccagtgtgattttgtttgacctttgtcttgacttaacgtcagcagggccaatttttatgtatttatgtaaatattgaaaaaaatgttggcacaattttaagacaaacacaaagggagattcttataaaggcttctcaggtggtgggcaagagttgggcaaaaaaatcaaggtatttggtcccggaacaaagct

SEQ ID NO: 2 - DNA, 2691 bp, Homo sapiens
  TATA_signal (2621)..(2627)
  CAAT_signal (2544)..(2552)
  protein_bind (2536)..(2539): c-Jun binding site
  protein_bind (2527)..(2530): c-Jun binding site
  protein_bind (2515)..(2518): c-Jun binding site
  misc_signal (2461)..(2475): Androgen response element (ARE)
  misc_signal (1549)..(1556): cAMP response element (CRE)
  protein_bind (1539)..(1545): Activator protein 1 (AP-1) response element
  ref <308>: DATABASE ACCESSION NUMBER: GenBank / AF151104
  ref <309>: DATABASE ENTRY DATE: 1999-08-23
  ref <313>: RELEVANT RESIDUES: (31)..(2721)
aagggactaagctcatagtaacttcacctggtaagtaacttttctttctgtttttattccagtaatgaaaaactgatagatgatttttagaaaaaaatgatcaaccttatctgaatatgtcacattcctggcctcagtatacgaacagcaattttgcagattagctgaatatggaaacaaaacgattttgtacatgatatcttcccttccctgtctgtaagcggattaaaattctactttaagaatacacacacagacacacacacacacacacacacaaaagatagactctggcaagaaattaaaatgttttgaggcccatgtctatacaccattagtgtaagtccttggtccaagttacttttttgttggtcttttaagtgagagacataaaaatggagttttattaattacttcttctctgtattttgagatataacatagtgctcccttcttctgctagcctgccaaacccatgttattgattttgatctcatcctgagaaatagtatagaaaaaaattacttcttatagtaaattactatagaaggtcaatacatcccacttattagtgttctagtttctttgagggaggctgaagtcagtcaaaatttatgcaattgggactcttgttatatcacctggaaatggcccggtttaatagaatttgtaagttcaaccagccatgtcagctccaaggtcaatgaaaagagtcttcatcattttacatcttcatttgttcattcatcctctcagcaaatatttatcaaaaacagtttttgttctggatgctagagataaaagggcaaagaagacacagtctcaacactcaaaagctgccagtctggcagataagtattctagattctggcttggttgggccctgtccacgttctttggcctgtggctgccggagaagcagaaggtcttgggttcatgagctcattttctttcttaatttgtgtagcagttattgcttataagatctatgtattgcgtttggtgggaggatttgataaaatgacccaatattgtccttttaaggagaaaattaatattagtaaagtttattgagagccagacaccatgctaaatagtttcaatgctttatctccttcctcatcagacctatgcaggagatcctcttagtcccatttcacagatgaggaaactgaggcctagagaagctcagttactttcttaaagtcacatgactagtcactgtggaaaagtctgggatctatctaagcttgtctactccaaagtgcctggtctggacccttatgtgatatttagaaatattaagtcatatttttatactttgaatatttcttcttctgtagcttgaccctatgaatggactctttagttgactatttttctagtaacaactcccaatttgtattactatttgtgtattaatacaattcttctacttctaattatggcactgaaatgacctctctcctccaaaaagtcatatgggatatacatttattaaatactcaaaatataatttttagggtatttctgtagaatgagtcattttgacgtcattccactcagctgtctttctgtattggcatcaaatttgctagatatgttgaaactgatgtaggagctatcgcagctatgctatattgcagtgctaagtcgcataactcagagctgcctcagattctctgttgagtagcacacgatttaatttgatgaaactgtatagatattttcaagtcaaaagtagcagtctttcagcaatgaatcatcattaagaattcatattttctaacaactcatcagtattctatcagtgagaactggattctaaaaagaacttactttatttttattttttattttttgagatggagtctcactctgtcgcccagctggaatgcagtggcatcatctctgctcactgcaacctccgcttcccagtttcaagagattcttctgcttcggcctcctgagtagctaggattacaggctcctgccactacgcccaactaatgttttgggttttttttgtatttttagtagagatggggtttcaccatgttgaccaggctggttttcaactcctgagctcaagtgatccgcttgcctcagcctcccaaagtgctaggattacagggatgagccactgtgcctggccaaaaagaagttattttagaatcaatctagatacccatccctccatacatatgaatcccaaaatacctataaatcatattgaatatctatttattaaatgtcaaacataggaatcctattattgtctctctttgaaaattaaggaaatattaactctaaattacacttaagacaaagtgtatacaagaaaactttttagggaagatttagaagattcaggcaaaatcatgagaaaaggtaaaatcagactctctaatgtatgggatacaaggagagaatgtacatagcccaaagtgcccagggcaaggtgaggtcagttcttaaattcctgattacattcagatccagtgtgattttgtttgacctttgtcttgacttaacgtcagcagggccaatttttatgtatttatgtaaatattgaaaaaaatgttggcacaattttaagacaaacacaaagggagattcttataaaggcttctcaggtggtgggcaagagttgggcaaaaaaatcaaggtatttggtcccggaacaaagct

SEQ ID NO: 3 - DNA, 483 bp, Homo sapiens
  misc_signal (415)..(429): Androgen response element (ARE)
  ref <308>: DATABASE ACCESSION NUMBER: GenBank / AF159056
  ref <309>: DATABASE ENTRY DATE: 2001-08-17
  ref <313>: RELEVANT RESIDUES: (93557)..(94039)
gatctggaaaacttgaactgaaatcctggctcagtttgaactccctaattctgtttcctcacatgacttttaatggtaataaccatttatagggtaaagcagaaagagataaatgcatagttagtgcctgactcatggtagacactatatttatgagttttcttctctttcactatcagaaagcacaggcccatggattgtcatcctgcagcagggggaaggtaagtgaaaaggagatttggggcttagagtagcatatcaatttctcaagacatttttccagacacgagtcttttatgcaaacatttcttgtttgccaaaaagaaacaagtgtttcaccaggcatgtttgcctgtgggagcagtatcagttgaaaagtatctataaatctacaatatgcatagtggcatgttaagtaccccaagtgcaaaagaaaccctaaaagactaaatcttcaggttaagaatgaagacaggtggcaca

SEQ ID NO: 4 - DNA, 875 bp, Homo sapiens
  misc_signal (611)..(625): Androgen response element (ARE)
  ref <308>: DATABASE ACCESSION NUMBER: GenBank / U37672
  ref <309>: DATABASE ENTRY DATE: 1996-03-24
  ref <313>: RELEVANT RESIDUES: (1067)..(1941)
ctgcagagaaattaattgtggcctgatgtccctgtcctggagagggtggaggtggaccttcactaacctcctaccttgaccctctcttttagggctctttctgacctccaccatggtactaggaccccattgtattctgtaccctcttgactctatgacccccactgcccactgcatccagctgggtcccctcctatctctattcccagctggccagtgcagtctcagtgcccacctgtttgtcagtaactctgaaggggctgacattttactgacttgcaaacaaataagctaactttccagagttttgtgaatgctggcagagtccatgagactcctgagtcagaggcaaaggcttttactgctcacagcttagcagacagcatgaggttcatgttcacattagtacaccttgccccccccaaatcttgtagggtgaccagagcagtctaggtggatgctgtgcagaaggggtttgtgccactggtgagaaacctgagattaggaatcctcaatcttatactgggacaacttgcaaacctgctcagcctttgtctctgatgaagatattatcttcatgatcttggattgaaaacagacctactctggaggaacatattgtatcgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggagaattgcctcccaacgctgttcagccagagccttccacccttgtctgcag

SEQ ID NO: 5 - DNA, 259 bp, Homo sapiens
  ref <308>: DATABASE ACCESSION NUMBER: GenBank / AF007544
  ref <309>: DATABASE ENTRY DATE: 1998-12-10
  ref <313>: RELEVANT RESIDUES: (14760)..(15018)
aattattttttcctttaacctttcaaactcaaggaaaaccagttggccttgactctgtttgtggaaaattttaaactactggtttaatttctttattggttgtaatatgactattttacgtcatataacaatttttattgtttgttaaatgactttattgtttgtcatatgataattttatgtcatagaacaatttttattgcttgatatatgactttattgttatatggctatacaactagatttttttgttgttttt

SEQ ID NO: 6 - DNA, 3126 bp, Mus musculus
  ref <308>: DATABASE ACCESSION NUMBER: GenBank / U19619
  ref <309>: DATABASE ENTRY DATE: 1998-06-29
  ref <313>: RELEVANT RESIDUES: (1852)..(4976)
  ref <308>: DATABASE ACCESSION NUMBER: GenBank / AF049091
  ref <309>: DATABASE ENTRY DATE: 2000-05-18
  ref <313>: RELEVANT RESIDUES: (2863)..(2921)
  ref <308>: DATABASE ACCESSION NUMBER: GenBank / AF049091
  ref <309>: DATABASE ENTRY DATE: 2000-05-18
  ref <313>: RELEVANT RESIDUES: (3636)..(3690)
tcatggaatgtattgcagtaccataatgcagaccccactaagcatggtcctcaaattctgcacatctatgaggacacctgacctccctagctccccaaagaatcacttaaggaaccgccaacaagaaagtctggtgatttgcgctttcgtattctaattggggcgttcagatgctaatgatctccggctcagggctgcaaacaattctgaaactgcattctctctcaatggggctcagctgccagcttcggataggaatcctacatcaagccttgggtgtcagccaaaatcaattgaagaggcggcaggacccctggctggtttgtggcagataatcgtacttatggcaaagttgggggttcacctgtttttgcactgtcccggagagccttgttaaaaggccaggaaggcaggacacctattagcccttccggaatggtctcctttggtctcagacccataaaccagtgcatgtggttcgatgccagaaagcacaaaagccgttgtgagtggaaagaccaaattgcttggcgctggtgactgtcatcttaaacattatgttccagagacagccaaagttaaggtttgcccatgacaatgtccaagggccaaagttcgggttcgcccacagcaatgtccgaagccgctatgcctcagtggtcgatatggtttataagaggttggaacacttgtgtttctggagggggtcctttggtcactgaaccccaaaaaccagccagtgtggctcactataggaaggcatagaagctgttatgtgcaacaagggaacggatgctaccgcgcggtggcagcatactcctatatatcgtggcccaaatgctgccaacttggggggagcgattcattcccagcaatatcccagggtcacccaaatagggattcataggggtggtaagatgtgtgcacctctggaatggttcccttacacactgaaccagagaacttgactcattccctacacagcccgagatcgtcagtggctggtaagaccgaagttgccgagcagcgaaccagtgcagtccacatacttatcatagaggtgaccaaaaattgcggttcacctatggcaaactcatgggtcactcaaggcatagcattcaatgttcataagggtcatggggtggatacaacacacatttcttgggcgctccttcagtcttgcgcccttcacgatcgatcggttcactctccacgctgtgcagatttggctatagctaaatggacagacgatgccgcgtggtggcagtacaatactacatattgctcggcagacgcggcataggttggggtccgctgtgacaaagcttgagtaccccaggttcaacaaagggatcaggcatttgtgacactttacggaatggtccccttctgccttgggactcctcaagctagccagatctgttcaaccaaactcaatacagaatcggttataggcgggagacatagaaactgcgcgtgcgtgcgtccaccgaaaccccatagccataaaagcagaggctgggttcaaccattgcaatgtcccaggtaacctaggaactgtagcaagaagttgcaaagctgagattagagactaggagaagtcaggatccgcttagtttgaagtgaagcaggagggtgggtagcaagagggagcataggcggtgtggcatggtggggttgtatagcgtgggttatagcgagggggtataatggggaggtatagtggaagtatagtgggaggtatagtaggggtatagcgggggatatagagggagttatagtgggggtatagcgggggcatagcgggggcataatgggggacatagtggggggccatggcaggaggggcatagtcgggggtataatggggggtgtagtggggcggtatagtgtgggtatagtggggacatagcgggggtatagtgggggtatagcagggagggtatagtgggggtatagcagagagtatgggggccataggggaagtatggtggggggtatggcaaggggtatggtggggaggatgacaggggggatatagcaggggtgtagtggggaggatatagtgggaagtataggggagtatagcaagggtgatgtggggggtgatgggagacagagaccaactgccaccacaccagtgaatagcttacatgtgatgccaaccctcggcctttagtctgctgatgctgccagggttcttgaggctcattagaagatggggtcattcttttccatcttatataccgatctctgaaaaggatcatttgtttaagagcgcagagatagccacaaccaccacaaggtgacagcacagacacacattacagcgccaagataatagaggctggggtctatttatgaccaaattggaggtgtccgtcagtatcataacttagggtccacccacagcaatgaccagggcttttcacagagtaaaaatgaaggatcactaggagccagaaatgagtactcagctgacatgacgcactaggctgaggatctgccaaggtgctattgcactcacataggtgagaaccactgctgagtggtcatgactggtcagcccttgagtcctcctcccatctggagctccggtcacttccatctagagctgctctactctctggtgccctgatttgtggatgctgactctgtggatttcagctgcataactttcaacagaagctaagagcaatggtatacgcacgcacgtggctggagggacagggcatctctcctgtccccattctatcactgatagtagtacttcagtaggatagggagaccatactgtgccctccggttgactggggtgaccaagaccagactgtaagataactaataaaactcatcgcaagagagtcctgacaaacgtgacgactcacccagaaaagggacggtgaggagtgcccaaattagctactaccttgttgaaaccactgcggccagataaggtcagtcaagcagactaggacaaaggctgggggttagccactcgtaggctgttcatactccgtgggatagtatgagacccatgccctcaaattcccattgtggtcattatacaaactgagtaaagggcagcttcattggcatcctggggcaccaccgttctatgaaggg

SEQ ID NO: 7 - DNA, 15 bp, Artificial Sequence
  Consensus sequence of Androgen Response Element (ARE)
  misc_feature (7)..(9): a or g or c or t
  misc_signal (1)..(15): Androgen Response Element (ARE)
ggtacannntgttct

SEQ ID NO: 8 - DNA, 15 bp, Homo sapiens
  misc_signal (1)..(15): Androgen Response Element (ARE)
  ref <301>: AUTHORS: Yu DC, Sakamoto GT, Hendersson DR
  ref <302>: TITLE: Identification of the transcriptional regulatory sequences of human kallikrein 2 and their use in the construction of calydon virus 764, an attenuated replication competent adenovirus for prost
  ref <303>: JOURNAL: Cancer Research
  ref <304>: VOLUME: 59
  ref <305>: ISSUE: 7
  ref <306>: PAGES: 1498-1504
  ref <307>: DATE: 1999-04-01
  ref <308>: DATABASE ACCESSION NUMBER: GenBank / AF113169
  ref <309>: DATABASE ENTRY DATE: 1999-05-05
  ref <313>: RELEVANT RESIDUES: (12077)..(12091)
ggaacagcaagtgct

SEQ ID NO: 9 - DNA, 15 bp, Homo sapiens
  misc_signal (1)..(15): Androgen Response Element (ARE)
  ref <301>: AUTHORS: Yu DC, Sakamoto GT, Henderson DR
  ref <302>: TITLE: Identification of the transcriptional regulatory sequences of human kallikrein 2 and their use in the construction of calydon virus 764, an attenuated replication competent adenovirus for prost
  ref <303>: JOURNAL: Cancer Research
  ref <304>: VOLUME: 59
  ref <305>: ISSUE: 7
  ref <306>: PAGES: 1498-1504
  ref <307>: DATE: 1999-04-01
  ref <308>: DATABASE ACCESSION NUMBER: GenBank / AF113169
  ref <309>: DATABASE ENTRY DATE: 1999-05-05
  ref <313>: RELEVANT RESIDUES: (8427)..(8436)
ggaacatattgtatt

SEQ ID NO: 10 - DNA, 15 bp, Homo sapiens
  misc_signal (1)..(15): Androgen Response Element (ARE)
  ref <301>: AUTHORS: Cleutjens KB, van Eekelen CC, van der Korput HA, Brinkman AO, Trapman J
  ref <302>: TITLE: Two androgen response regions cooperate in steroid hormone regulated activity of the prostate-specific antigen promoter
  ref <303>: JOURNAL: Journal of Biological Chemistry
  ref <304>: VOLUME: 271
  ref <305>: ISSUE: 11
  ref <306>: PAGES: 6379-6388
  ref <307>: DATE: 1996-03-15
  ref <308>: DATABASE ACCESSION NUMBER: GenBank / X92553
  ref <309>: DATABASE ENTRY DATE: 1996-04-12
  ref <313>: RELEVANT RESIDUES: (463)..(477)
agaacagcaagtgct

SEQ ID NO: 11 - DNA, 15 bp, Homo sapiens
  misc_signal (1)..(15): Anrogen Response Element (ARE)
  ref <301>: AUTHORS: Schuur ER, Henderson GA, Kmetec LA, Miller JD, Lamparski HG, Henderson DR
  ref <302>: TITLE: Prostate-specific antigen expression is regualted by an upstream enhancer
  ref <303>: JOURNAL: Journal of Biological Chemistry
  ref <304>: VOLUME: 271
  ref <305>: ISSUE: 12
  ref <306>: PAGES: 7043-7051
  ref <307>: DATE: 1996-03-22
  ref <308>: DATABASE ACCESSION NUMBER: GenBank / U37672
  ref <309>: DATABASE ENTRY DATE: 1996-03-24
  ref <313>: RELEVANT RESIDUES: (1677)..(1691)
ggaacatattgtatc

SEQ ID NO: 12 - DNA, 15 bp, Rattus sp.
  misc_signal (1)..(15): Androgen Response Element (ARE)
  ref <301>: AUTHORS: Rennie PS, Bruchovsky N, Leco KJ, Sheppard PC, McQueen SA, Cheng H, Snoek R, Hamel A, Bock ME, MacDonald BS, et al.
  ref <302>: TITLE: Characterization of two cis-acting DNA elements involved in the androgen regulation of the probasin gene
  ref <303>: JOURNAL: Molecular Endocrinology
  ref <304>: VOLUME: 7
  ref <305>: ISSUE: 1
  ref <306>: PAGES: 23-36
  ref <307>: DATE: 1993-01
  ref <308>: DATABASE ACCESSION NUMBER: GenBank / S56983
  ref <309>: DATABASE ENTRY DATE: 1993-06-28
  ref <313>: RELEVANT RESIDUES: (291)..(305)
agtactccaagaacc

SEQ ID NO: 13 - DNA, 15 bp, Rattus sp.
  misc_signal (1)..(15): Androgen Response Element (ARE)
  ref <301>: AUTHORS: Rennie PS, Bruchovsky N, Leco KJ, Sheppard PC, McQueen SA, Cheng H, Snoek R, Hamel A, Bock ME, MacDonald BS, etal.
  ref <302>: TITLE: Characterization of two cis-acting DNA elements involved in the androgen regulation of the probasin gene
  ref <303>: JOURNAL: Molecular Endocrinology
  ref <304>: VOLUME: 7
  ref <305>: ISSUE: 1
  ref <306>: PAGES: 23-36
  ref <307>: DATE: 1993-01
  ref <308>: DATABASE ACCESSION NUMBER: GenBank / S56983
  ref <309>: DATABASE ENTRY DATE: 1993-06-28
  ref <313>: RELEVANT RESIDUES: (186)..(200)
atagcatcttgttct

SEQ ID NO: 14 - DNA, 15 bp, Mus musculus
  misc_signal (1)..(15): Androgen Response Element (ARE)
  ref <301>: AUTHORS: Fabre S, Manin M, Pailhoux E, Veyssiere G, Jean C
  ref <302>: TITLE: Identification of a functional androgen response element in the promoter of the gene for the androgen-regulated aldose reductase-like protein specific to the mouse vas deferens
  ref <303>: JOURNAL: Journal of Biological Chemistry
  ref <304>: VOLUME: 269
  ref <305>: ISSUE: 8
  ref <306>: PAGES: 5857-5864
  ref <307>: DATE: 1994-02-25
  ref <308>: DATABASE ACCESSION NUMBER: GenBank / U00149
  ref <309>: DATABASE ENTRY DATE: 1994-04-20
  ref <313>: RELEVANT RESIDUES: (1694)..(1708)
tgaagttcctgttct

SEQ ID NO: 15 - DNA, 15 bp, Homo sapiens
  misc_signal (1)..(15): Androgen Response Element (ARE)
  ref <308>: DATABASE ACCESSION NUMBER: GenBank / AF 151104
  ref <309>: DATABASE ENTRY DATE: 1999-08-23
  ref <313>: RELEVANT RESIDUES: (2491)..(2505)
ggtgaggtcagttct

SEQ ID NO: 16 - DNA, 15 bp, Homo sapiens
  misc_signal (1)..(15): Androgen Response Element (ARE)
  ref <308>: DATABASE ACCESSION NUMBER: GenBank / AF159056
  ref <309>: DATABASE ENTRY DATE: 2001-08-17
  ref <313>: RELEVANT RESIDUES: (93971)..(93985)
agtaccccaagtgca

SEQ ID NO: 17 - DNA, 23 bp, Artificial Sequence
  Syntetic oligonucleotide comprising sequence of Androgen Response Element (ARE) of TARP promoter
  misc_signal (5)..(19): Androgen Response Element (ARE)
gcaaggtgaggtcagttcttaaa

SEQ ID NO: 18 - DNA, 23 bp, Artificial Sequence
  Syntetic oligonucleotide comprising mutated sequence of Androgen Response Element (ARE) of TARP promoter
gcaaggtgtggtcaactattaaa